(19) United States
(12) Patent Application Publication
(10) Pub. No.: US 2025/0186069 A1
(43) Pub. Date: Jun. 12, 2025

(54) ADAPTIVE ASPIRATION BASED ON CLOT DETECTION IN THROMBECTOMY DEVICES

(71) Applicant: Walk Vascular, LLC, Irvine, CA (US)

(72) Inventors: Matthew J. GILLICK, Murrieta, CA (US); Nianjiong BEI, Palo Alto, CA (US); Seung Hae Clare CHUNG, Los Altos, CA (US); Grant A. ADAMS, New Brighton, MN (US)

(21) Appl. No.: 18/968,880

(22) Filed: Dec. 4, 2024

Related U.S. Application Data
(60) Provisional application No. 63/607,451, filed on Dec. 7, 2023.

(51) Int. Cl. A61B 17/22; A61B 17/00; A61B 90/00; G16H 40/63

(52) U.S. Cl. CPC A61B 17/22; A61B 17/00234; A61B 90/06; G16H 40/63; A61B 2017/00022; A61B 2017/00238; A61B 2017/00561; A61B 2017/22079; A61B 2090/064; A61B 2217/005; A61B 2217/007

(57) ABSTRACT

A thrombectomy system is configurable to (i) activate a low-level aspiration state, wherein the low-level aspiration state comprises operation of one or more aspiration components; (ii) during operation of the low-level aspiration state, monitor sensor data associated with at least some of the one or more aspiration components; (iii) process the sensor data using a clot engagement analysis module; and (iv) after determining that output of the clot engagement analysis module satisfies one or more conditions, selectively activate a clot removal state, wherein the clot removal state comprises operation of the one or more aspiration components with different parameters than the low-level aspiration state.

### CROSS-REFERENCE TO RELATED APPLICATION(S)

This application claims priority to U.S. Provisional Patent Application No. 63/607,451, filed on Dec. 7, 2023, and entitled “ADAPTIVE ASPIRATION BASED ON CLOT DETECTION IN THROMBECTOMY DEVICES”, the entirety of which is incorporated herein by reference for all purposes.

### BACKGROUND

### Technical Field

The present disclosure pertains generally to medical devices and methods of their use. More particularly, the present invention pertains to aspiration and thrombectomy devices and methods of use thereof.

### Description of the Related Technologies

Blood clots (thrombi) can form in various parts of the body and can pose a serious health risk. For instance, blood clots can block blood flow and/or lead to tissue damage, organ dysfunction, or life-threatening conditions like stroke or heart attack. Thrombectomy is a medical procedure used to remove blood clots from blood vessels to restore blood flow and prevent further complications.

Various types of catheter-based thrombectomy devices have been developed to aid in the removal of thrombotic material. Such devices are typically inserted into the affected blood vessel through a small incision or artery access point. Catheter-based thrombectomy devices include mechanical thrombectomy devices, rheolytic thrombectomy devices, and others (e.g., ultrasound-assisted devices).

Mechanical thrombectomy devices can implement various types of mechanical components to engage with and remove thrombotic material. For instance, stent retrievers and clot retriever baskets are designed for navigation through vasculature to the site of a clot, deployment at the clot site to cause the stent retriever or clot retriever basket to entrap the clot, and withdrawal through the vasculature to facilitate clot removal. As another example, suction-based thrombectomy devices use negative pressure to aspirate clots from blood vessels (e.g., via a catheter with a distal tip to be placed near the clot prior to activation to draw the clot into a collection chamber, where it is trapped and removed). As yet another example, rotational thrombectomy devices employ rotational mechanisms to fragment and remove clots (e.g., a rotating wire or catheter tip for creating shear forces that break down clots), allowing the fragments to be cleared by the body or using aspiration or other techniques.

Rheolytic thrombectomy devices employ mechanisms that rely on high-velocity jets to break down and remove thrombotic material. Rheolytic thrombectomy mechanisms may be positioned on catheters (e.g., at or near the distal tip) and can utilize saline solution, or a mixture of saline and the patient's own blood, to create high-velocity jets for direction toward clots to generate shear forces that disrupt the clot's structure. The jetted fluid can cause fragmentation of the clot, and the fragments may then be cleared naturally from the body or by aspiration techniques.

Some thrombectomy devices employ aspects of suction-based thrombectomy devices and rheolytic thrombectomy devices. For instance, some thrombectomy devices utilize a saline jet positioned at or near a distal tip of an aspiration catheter, allowing for aspiration of clot fragments as the jetted saline macerates the clot (e.g., thrombus and/or soft emboli).

To facilitate clot removal, catheter-based thrombectomy devices typically need to be navigated toward a clot site within patient vasculature. However, users (e.g., healthcare practitioners) often experience difficulty in determining whether a catheter of a thrombectomy device is appropriately positioned to perform clot removal functions. Users often resort to a trial-and-error approach, which can result in excessive patient blood loss (e.g., in systems that implement aspiration), damage to vessel walls, increased treatment times (which can give rise to other patient risks), failed clot removal, and/or other negative outcomes for patients. Furthermore, a lack of visualization of clot engagement can make it difficult for users to determine when clot removal is complete, which can also lead to excessive patient blood loss (e.g., when aspiration processes persist after clot removal), damage to vessel walls (e.g., from repeated clot removal process activations/de-activations), increased treatment times, failed clot removal, and/or other negative outcomes for patients.

The subject matter disclosed herein is not limited to embodiments that operate only in environments such as those described above. Rather, this background is only provided to illustrate one example technology area where some embodiments described herein may be practiced.

### SUMMARY

At least some disclosed embodiments provide a thrombectomy system that is configurable to (i) activate a low-level aspiration state, wherein the low-level aspiration state comprises operation of one or more aspiration components; (ii) during operation of the low-level aspiration state, monitor sensor data associated with at least some of the one or more aspiration components; (iii) process the sensor data using a clot engagement analysis module; and (iv) in response to determining that output of the clot engagement analysis module satisfies one or more conditions, selectively activate a clot removal state, wherein the clot removal state comprises operation of the one or more aspiration components with different parameters than the low-level aspiration state.

At least some disclosed embodiments provide a thrombectomy system that is configurable to (i) during operation of a clot removal state that comprises operation of one or more aspiration components, monitor sensor data associated with at least some of the one or more aspiration components; (ii) process the sensor data using a clot removal analysis module; and (iii) in response to determining that output of the clot removal analysis module satisfies one or more conditions, selectively deactivate the clot removal state.

At least some disclosed embodiments provide a thrombectomy system that is configurable to (i) during operation of a low-level aspiration state or a clot removal state that comprises operation of one or more aspiration components, monitor flow data obtained by a flow sensor associated with the thrombectomy system; (ii) (iii) process the flow data using a flow data analysis module; and (iv) in response to determining that output of the flow data analysis module satisfies one or more conditions, selectively deactivate the low-level aspiration state or the clot removal state.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates a perspective view of an example aspiration catheter.

FIG. 2 illustrates a plan view of example disposable components of a system for aspirating thrombus according to an embodiment of the present disclosure.

FIG. 3 illustrates a sectional view of an example distal end of the aspiration catheter of the system for aspirating thrombus of FIG. 1.

FIG. 4 illustrates a detail view of an example y-connector of the aspiration catheter of the system for aspirating thrombus of FIG. 1.

FIG. 5 illustrates a plan view of example disposable components of a system for aspirating thrombus according to an embodiment of the present disclosure.

FIG. 6A illustrates a perspective view of an example system for aspirating thrombus of FIG. 4.

FIG. 6B illustrates a schematic representation of the aspiration system according to an implementation of the present disclosure.

FIG. 7 illustrates a conceptual representation of determining clot engagement status during a clot removal state of a thrombectomy system based on sensor data, according to implementations of the present disclosure.

FIGS. 8A, 8B, and 9 illustrate example sensor data that may be used to determine clot engagement status of a thrombectomy system, according to implementations of the present disclosure.

FIG. 10 illustrates a conceptual representation of controlling a clot removal state based on flow data, in accordance with implementations of the present disclosure.

FIG. 11 illustrates a conceptual representation of determining clot engagement status during a low-level aspiration state of a thrombectomy system based on sensor data, according to implementations of the present disclosure.

FIG. 12 illustrates a conceptual representation of controlling a low-level aspiration state based on flow data, in accordance with implementations of the present disclosure.

FIGS. 13-16 illustrate example flow diagrams depicting acts associated with controlling operation of a thrombectomy system based on sensor data, in accordance with implementations of the present disclosure.

FIG. 17 depicts graphs that provide conceptual flow rate and pressure signals associated with operation of a thrombectomy system, in accordance with implementations of the present disclosure.

FIGS. 18 and 19 depict graphs that provide conceptual pressure signals associated with operation of a thrombectomy system, in accordance with implementations of the present disclosure.

### DETAILED DESCRIPTION

As indicated hereinabove, many conventional catheter-based thrombectomy devices lack mechanisms to detect whether the catheter is engaged with a clot or not. For example, many aspiration catheters lack a mechanism to determine whether the distal end of the aspiration catheter is engaged with a clot. Users thus often resort to trial and error or other methods to determine when to activate or de-activate aspiration or other clot removal functionalities, which can lead to in excessive patient blood loss (e.g., in systems that implement aspiration), damage to vessel walls, increased treatment times (which can give rise to other patient risks), failed clot removal, and/or other negative outcomes for patients.

The present disclosure pertains to systems, devices, and techniques for detecting clot engagement in thrombectomy systems.

For example, a thrombectomy system may implement signal-based clot detection by, during operation of a clot removal state, monitoring sensor data associated with operation of aspiration components (or other clot removal components). The sensor data may be processed (e.g., by comparison to reference data, or by using the sensor data as input to one or more AI modules) to determine whether the sensor data indicates that the aspiration catheter is engaged with a clot. If the sensor data indicates that the aspiration catheter is engaged with a clot, the thrombectomy system may selectively remain in the clot removal state. In contrast, if the sensor data indicates that the aspiration catheter is not (or is no longer) engaged with a clot, the thrombectomy system may selectively deactivate the clot removal state and/or present an alert to the user.

In some implementations, thrombectomy systems may implement adaptive aspiration based on clot detection to achieve further advantages. For example, a thrombectomy system may be configured to operate in a low-level aspiration state, where aspiration components are operated at a lower setting as compared to a clot removal state. During operation of the low-level aspiration state, sensor data associated with the aspiration components may be monitored and/or processed to determine whether the aspiration catheter is engaged with a clot (or not). In response to determining, based on the sensor data, that the aspiration catheter is not engaged with a clot, the thrombectomy system may remain in the low-level aspiration state. In contrast, in response to determining, based on the sensor data, that the aspiration catheter is engaged with a clot, the thrombectomy system may selectively enter a clot removal state (e.g., by increasing the setting of the aspiration components).

After transitioning to the clot removal state (e.g., a full aspiration state), the thrombectomy system may continue to monitor sensor data to determine whether the sensor data indicates that clot removal is completed, which may trigger automatic de-activation of the clot removal state (e.g., reverting back to the low-level aspiration state and/or fully de-activating aspiration).

In some implementations, a thrombectomy system is configured to monitor flow data during operation of a clot removal state or low-level aspiration state. The flow data may be processed (e.g., by comparison to reference data) to determine whether the flow data indicates that blood loss by the patient is approaching (or is likely to approach) dangerous levels. In response to determining that the flow data satisfies such conditions, the thrombectomy system may selectively deactivate the clot removal state or the low-level aspiration state.

In some instances, implementing signal-based clot detection and/or adaptive aspiration on a thrombectomy device, as described herein, may provide various advantages, such as minimized blood loss while attempting to engage with a clot, minimized blood loss after completing clot aspiration, minimized incidence of excessive patient blood loss, providing user feedback as to when a clot is detected, confirming whether a clot is present after aspiration, verifying vessel clearance, etc. Such advantages can beneficially facilitate improved patient outcomes.

Although examples discussed herein focus, in at least some respects, on implementing signal-based clot detection and/or adaptive aspiration on an aspiration catheter, the techniques and/or components discussed herein may be implemented on other types of catheter-based devices, even outside of the domain of thrombectomy.

### Example Thrombectomy Device

The following discussion relates to an example catheter-based thrombectomy device that implements aspiration aspects and rheolytic aspects and that may comprise or be used to implement at least some disclosed embodiments. As noted above, the principles disclosed herein may be implemented in conjunction with other types of catheter-based thrombectomy systems.

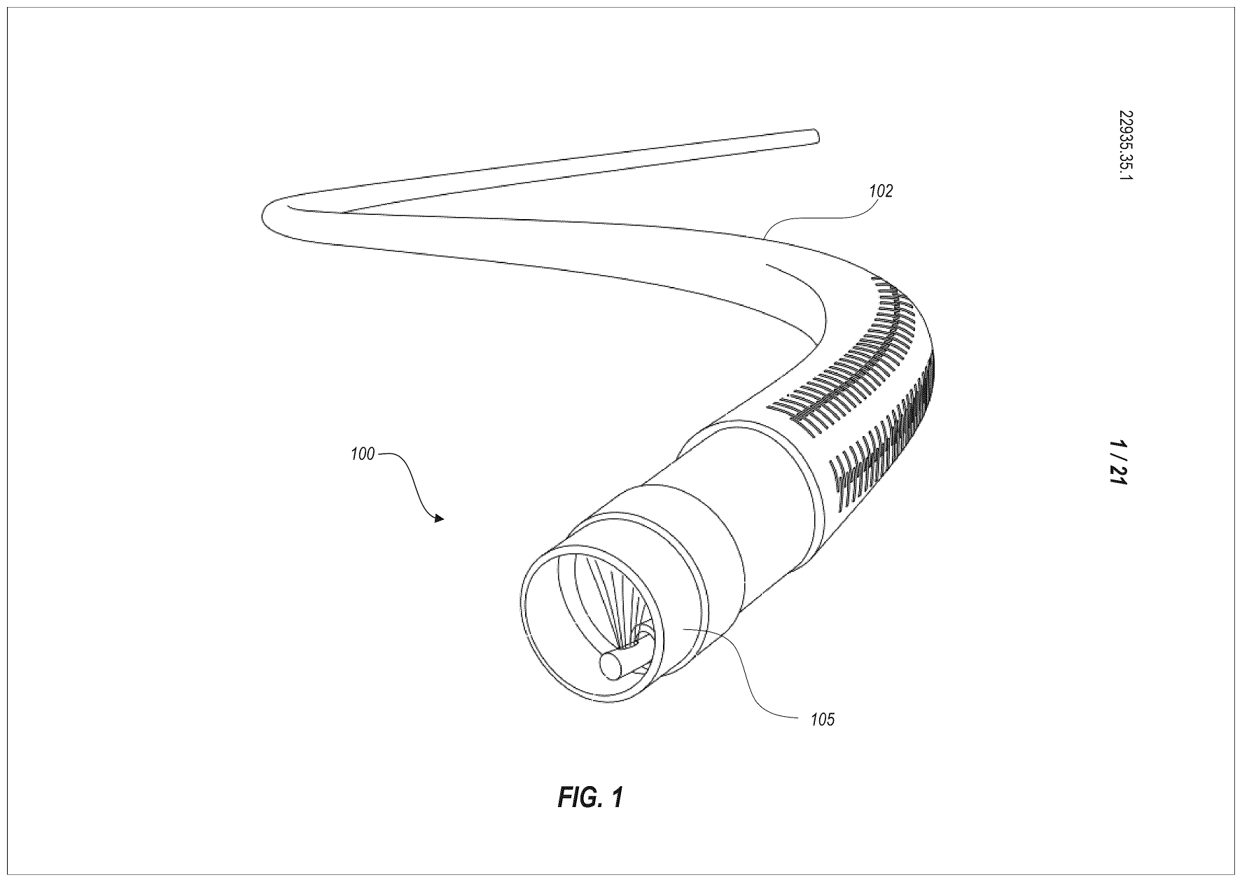

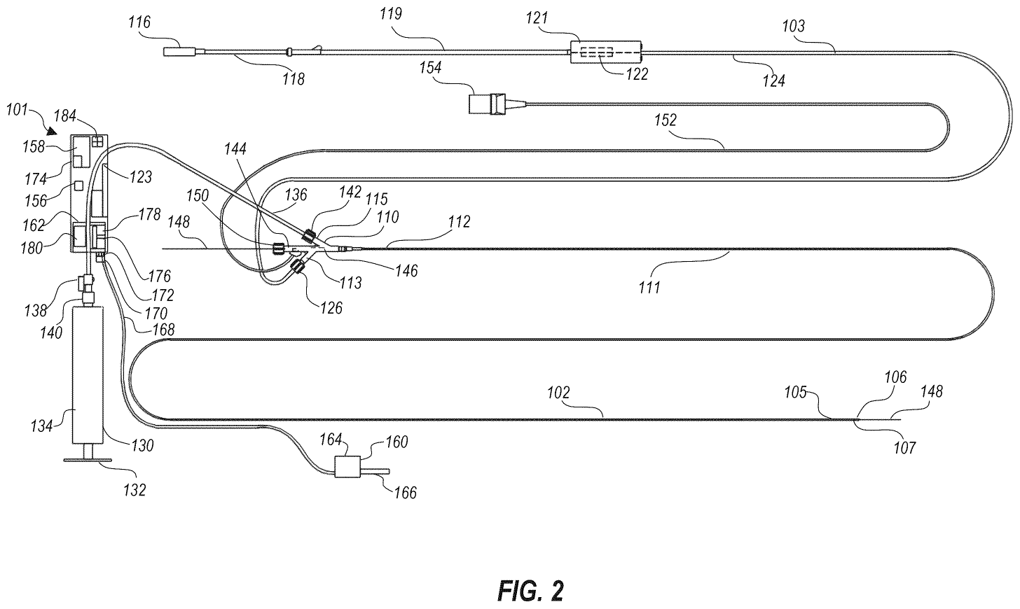

A system 100 for aspirating thrombus is illustrated in FIG. 1, illustrating primarily a distal end 105 of an aspiration catheter 102. FIGS. 2-4 illustrate the system 100 in greater detail. The system 100 for aspirating thrombus includes three major components: a pump 101, an aspiration catheter 102, and a tubing set 103. The aspiration catheter 102 and the tubing set 103 may comprise disposable components. The pump 101 and the pump's associated pump base may comprise reusable components. In some implementations, it is not necessary to sterilize the pump 101, as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

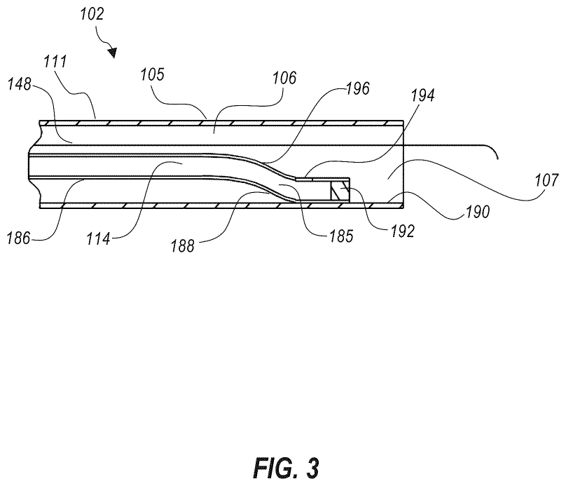

As shown in FIGS. 2-4, the aspiration catheter 102 has a distal end 105 and includes an over-the-wire guidewire lumen/aspiration lumen 106 extending between an open distal end 107, and a proximal end comprising a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 comprises a first female luer 113 which communicates with a catheter supply lumen 114 (FIG. 3), and a second female luer 115 which communicates with the guidewire lumen/aspiration lumen 106.

A spike 116 for coupling to a fluid source (e.g., saline bag, saline bottle) allows fluid to enter through an extension tubing 118 and flow into a supply tube 119. An optional injection port allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

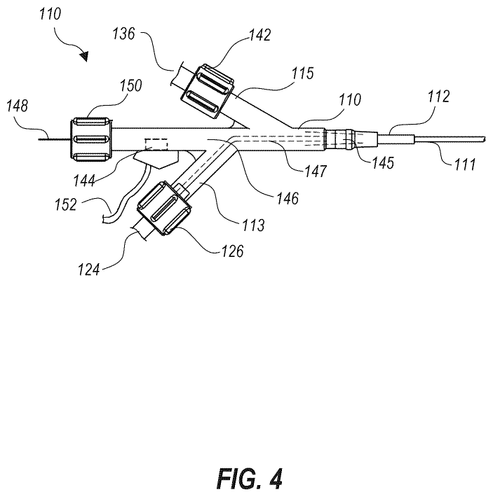

Accessories are illustrated that are intended for applying a vacuum source, such as a syringe 130 having a plunger 132 and a barrel 134, to the aspiration lumen 106 of the catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 4, a pressure sensor or transducer 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector 154. The connector 154 is plugged into a socket 156 of the pump 101. Pressure related signals may be processed by a circuit board 158 of the pump 101. The pressure transducer 144 may be powered from the pump 101, via the cable 152. The accessories may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure transducer 144 thus senses a negative pressure and sends a signal, causing the controller to start the motor of the pump 101. As the effect via the electronics is substantially immediate, the motor starts pumping almost immediately after the pedal 166 is depressed. As the pedal 166 of the foot pedal 160 is released, the controller 174 then causes the pinch valve 162 to close. The pressure transducer 144 thus senses that no negative pressure is present and the controller 174 causes the motor of the pump 101 to shut off. Again, the effect via the electronics is substantially immediate, and thus the motor stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184 or other user interfaces.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 3, a supply tube 186, which contains the catheter supply lumen 114, freely and coaxially extends within the over-the-wire guidewire lumen/aspiration lumen 106. At least a distal end 188 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 188 of the supply tube 186. The plug 192 blocks the exit of pressurized fluid, and thus the pressurized fluid is forced to exit through an orifice 194 in the wall 196 of the supply tube 186 (forming a fluid jet, such as a saline jet). The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torquability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

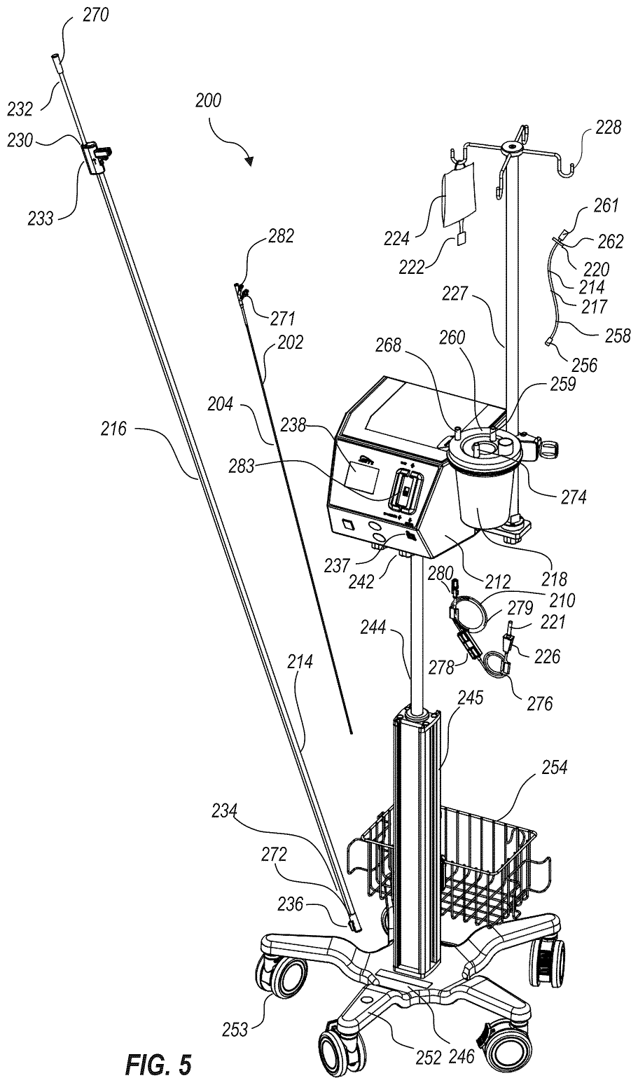

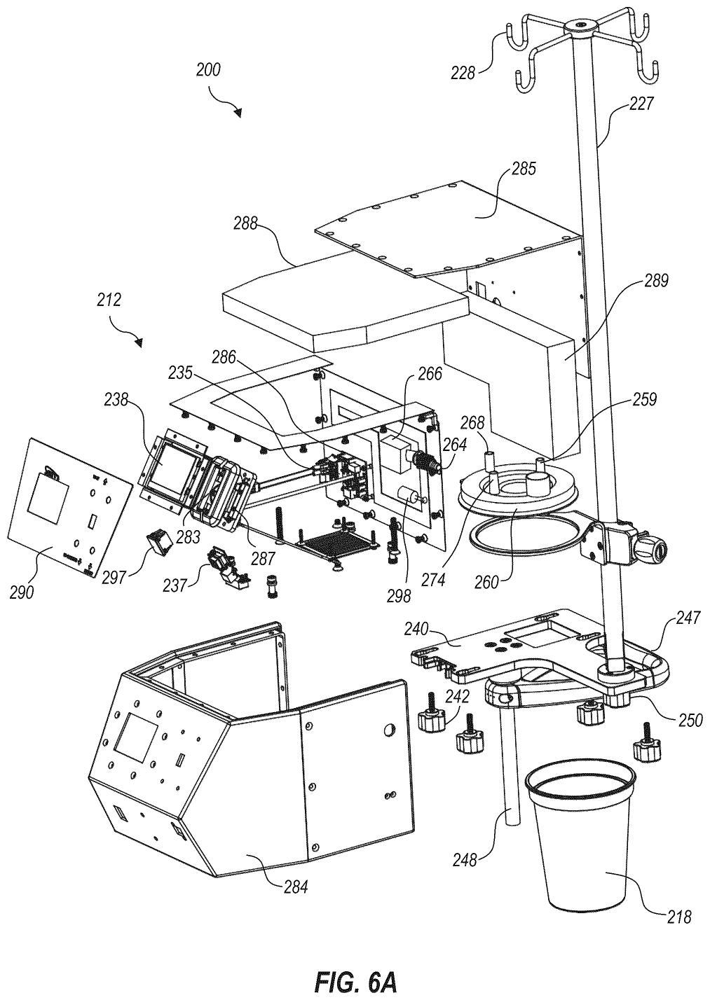

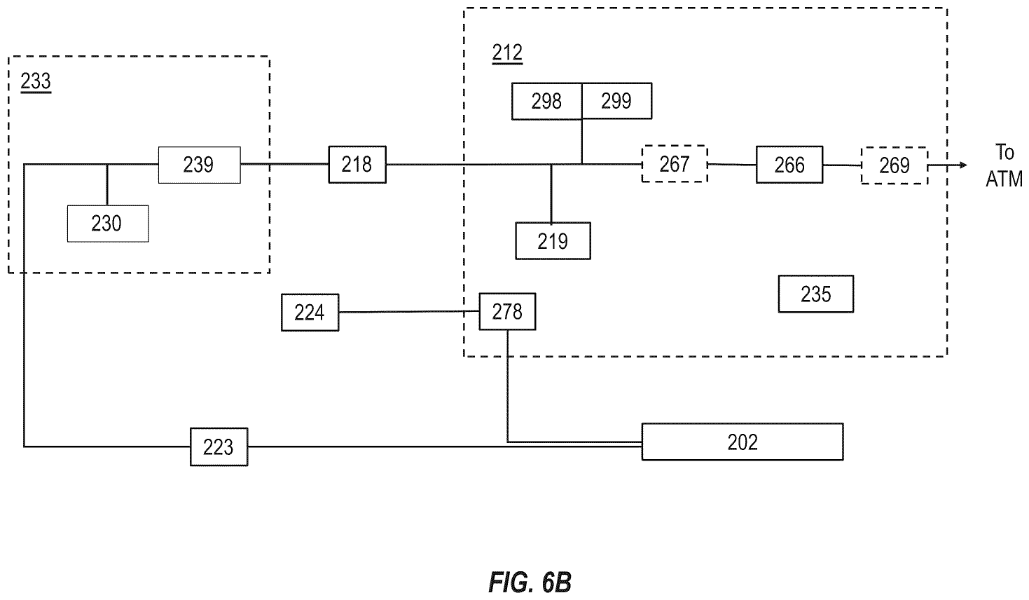

A system 200 for aspirating thrombus is illustrated in FIGS. 5 and 6A. FIG. 6B is schematically illustrated with functional block associated with the functions of structures described herein. An aspiration catheter 202 is similar to the aspiration catheter 102 of FIGS. 1-4. The aspiration catheter 202 is configured for aspirating thrombus from peripheral vessels, but may also be configured with a size for treating coronary, cerebral, pulmonary or other arteries, or veins. The aspiration catheter 202/system 200 may be used in interventional procedures, but may also be used in surgical procedures. The aspiration catheter 202/system 200 may be used in vascular procedures, or non-vascular procedures (other body lumens, ducts, or cavities). The catheter 202 comprises an elongate shaft 204 configured for placement within a blood vessel of a subject. The catheter 202 may also comprise a catheter supply lumen 114 (FIG. 3) and a guidewire/aspiration lumen 106, each extending along the shaft. The supply lumen 114 may have a proximal end 147 and a distal end 185, and the aspiration lumen 106 may have a proximal end 145 (FIG. 4) and an open distal end 107 (FIG. 3). An orifice or opening 194 may exist at or near the distal end 185 of the supply lumen 114. The orifice or opening 194 may be configured to allow the injection of pressurized fluid into the aspiration lumen 106 at or near the distal end 107 of the aspiration lumen 106 when the pressurized fluid is pumped through the supply lumen 114. In some embodiments, the orifice or opening 194 may be located proximal to the distal end 185 of the supply lumen 114. In some embodiments, the distal end 185 of the supply lumen 114 may comprise a plug 192.

Although examples provided herein focus, in at least some respects, on an aspiration catheter 102 that includes a single orifice 194, an aspiration catheter of a system (e.g., 100 or 200) can include any quantity of orifices through which fluid may pass to form any quantity of fluid jets. For example, a system 100 or 200 for aspirating thrombus can include a quantity of orifices (for forming fluid jets) within a range of 1 to 10, or within a range of 2 to 8, or within a range of 3 to 6.

Although FIG. 3 illustrates the orifice 194 formed in the wall 196 of the supply tube 186, an orifice for forming a fluid jet for an aspiration catheter of a system (e.g., 100 or 200) can be positioned on one or more additional components that are in fluid communication with the supply tube 186. By way of illustrative example, one or more orifices may be formed on a ring (or annulus) that is connected to the catheter shaft 111 (e.g., coaxially connected to the catheter shaft 111 at or near the open distal end 107). The ring can include an internal cavity in fluid communication with the supply tube 186, allowing fluid to travel through the supply tube 186 and the internal cavity of the ring for jetting through the orifice(s) of the ring.

Furthermore, FIG. 3 illustrates the orifice 194 formed as a radial opening in the wall 196 of the supply tube 186, with the orifice wall(s) that forms the orifice 194 between inner and outer surfaces of the supply tube 186 being perpendicular to the tangential axis of the wall 196. In some embodiments, an orifice for forming a fluid jet for an aspiration catheter of a system (e.g., 100 or 200) may be formed as an angled opening, where the orifice wall(s) that forms the orifice (between inner and outer surfaces of a material) has an acute or obtuse angle relative to the tangential axis of the material on which the orifice is formed (e.g., material of a supply tube or an additional component connected to the supply tube). For example, the angle between the orifice wall(s) and the tangential axis of the material on which the orifice is formed can be within a range of 20° to 70°, or 30° to 60°, or 40° to 50° (or within their complementary ranges).

The orifice(s) of an aspiration catheter 102 of a system (e.g., 100 or 200) for providing fluid jets (i.e., “jet-forming” orifices) may be implemented with various sizes or shapes in different embodiments. In one example, an orifice may comprise a circular hole with a diameter between 0.03 mm (0.001 inches) and 0.15 mm (0.006 inches), or between about 0.0508 mm (0.002 inches) and about 0.1016 mm (0.004 inches), or about 0.0787 mm (0.0031 inches). The diameter of the supply lumen 114 may be between about 0.3048 mm (0.012 inches) and about 0.4826 mm (0.019 inches), or between about 0.3556 mm (0.014 inches and about 0.4318 mm (0.017 inches) or about 0.3937 mm (0.0155 inches). As another example, an orifice may comprise a rectangular hole with each side thereof having a length between 0.02 mm (0.0008 inches) and 0.20 mm (0.008 inches). In some implementations, the total cross-sectional area of all jet-forming orifices of an aspiration catheter of a system is between 0.002 mm\hat{ }2 and 0.02 mm\hat{ }2, or between 0.003 mm\hat{ }2 and 0.015 mm\hat{ }2, or between 0.005 mm\hat{ }2 and 0.01 mm\hat{ }2. In some embodiments, pressure measured at the deliver location of each jet-forming orifice during operation of the system (e.g., 100 or 200) is between 400 psi (2.6 MPa) and 2,000 psi (13.8 MPa), or between 500 psi (3.4 MPa) and 2,000 psi (13.8 MPa), or between 600 psi (4.1 MPa) and 1,750 psi (12.1 MPa).

In some implementations, the diameter of the aspiration lumen 106 is within a range of 2 mm (0.08 inches) to 4 mm (0.16 inches). The orifice 194 may be set proximally of the open distal end 107 by a set amount. For example, orifice 194 can be set proximally of the open distal end 107 by about 0.040″ (inches), and in one configuration by 0.051″ +−0.003″ or by another desired amount. For example, orifice 42 can be set proximally of the open distal end 107 by approximately 0.035″, 0.036″, 0.037″, 0.038″, 0.039″, 0.040″, 0.041″, 0.042″, 0.043″, 0.044″, 0.045″, 0.046″, 0.047″, 0.048″, 0.049″, 0.050″, 0.051″, 0.052″, 0.053″, 0.054″, 0.055″, 0.056″, 0.057″, 0.058″, 0.059″, 0.060″, or a range defined by any two of the foregoing. In still other configurations, the open distal end 107 can be set proximally of the open distal end 107 by about 0.01″ to about 50″.

In still another configuration a diameter of the aspiration lumen 106 is about 0.075″ (inches) to about 0.177″ (inches), the orifice 194 (and so a distal end of the supply tube 186) can be set proximally of open distal end 107 about 12″ (inches). In still another configuration a diameter of the aspiration lumen 106 is about 0.075″ (inches), the orifice 194 (and so a distal end of the supply tube 186) can be set proximally of open distal end 107 about 0.035″ (inches) to about 0.060″ (inches). In still another configuration, the orifice 194 (and so a distal end of the supply tube 186) can be set proximally of open distal end 107 about 0.010″ (inches) to about 50″ (inches) where the catheter 102 has a catheter size ranging from about 3 Fr to about 50 Fr. In still another configuration, the orifice 194 (and so a distal end of the supply tube 186) can be set proximally of open distal end 107 about 0.010″ (inches) to about 0.200″ (inches), from about 0.010″ (inches) to about 2″ (inches), 0.010″ (inches) to about 40″ (inches), 0.010″ (inches) to about 50″ (inches), or a range defined by any two of the foregoing. In still other configurations, the orifice 194 (and so a distal end of the supply tube 186) can be set proximally of open distal end 107 based upon Table 1:

TABLE 1

| Catheter size | Distal Orifice Proximal Offset (range in inches) |
|---|---|
| 3 Fr | .01″ to .600″ |
| 4 Fr | |
| 5 Fr | |
| 6 Fr | |
| 7 Fr | |
| 8 Fr | |
| 9 Fr | 0.01″ to 40″ |
| 10 Fr | |
| 11 Fr | |
| 12 Fr | .01″ to 40″ |
| 13 Fr | |
| 14 Fr | |
| 15 Fr | |
| 16 Fr | |
| 17 Fr | .01″ to 50″ |
| 18 Fr | |
| 19 Fr | |
| 20 Fr | |
| 21 Fr | |
| 22 Fr | |
| 23 Fr | |
| 24 Fr | |
| 25 Fr | |
| 26 Fr | |

In another configuration, the orifice 194 (and so a distal end of the supply tube 186) can be set proximally of open distal end 107 about 0.010″ (inches) to about 0.200″ (inches) for a catheter having a catheter size ranging from about 3 Fr to about 8 F. In another configuration, the orifice 194 (and so a distal end of the supply tube 186) can be set proximally of open distal end 107 about 0.010″ (inches) to about 2″ (inches) for a catheter having a catheter size ranging from about 9 Fr to about 11 Fr. In another configuration, the orifice 194 (and so a distal end of the supply tube 186) can be set proximally of open distal end 107 about 0.010″ (inches) to about 40″ (inches) for a catheter having a catheter size ranging from about 12 Fr to about 16 F. In another configuration, the orifice 194 (and so a distal end of the supply tube 186) can be set proximally of open distal end 107 about 0.010″ (inches) to about 50″ (inches) for a catheter having a catheter size ranging from about 17 Fr to about 26 F. In other configurations, the ranges can include any combination of a range defined by any two of the foregoing proximal location of the orifice 194 with a range defined by any two of the foregoing catheter sizes.

A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. Additional or alternative pressure sensors may be implemented to measure pressure associated with the vacuum canister 218. The pressure sensor 230 is shown in FIG. 5 within a lumen at a junction between a first aspiration tube 232 and a control 233. The control 233 can include an operable valve 239 (see FIG. 6B) through which fluid flows to the supply lumen 114. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In some instances, one or more fluid flow sensors is/are utilized in addition to or as an alternative to the pressure sensor 230. In some embodiments, the fluid flow sensor is a Doppler flow velocity sensor, or other type of flow sensor. In one example, a flow sensor 223 is positioned along tubing between the aspiration catheter 202 and the vacuum canister 218 (see FIG. 6B). In some instances, flow metrics may be inferred or characterized by implementing multiple pressure sensors (e.g., (i) a pressure sensor on the pump set 210, suction tubing 214, or aspiration lumen 106, and (ii) a pressure sensor on the vacuum canister 218).

In the example of FIGS. 5 and 6A, the SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a second port 259 on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. The vacuum pump 266 communicates with atmosphere through a manifold and/or filter 269 (see FIG. 6B). In some instances, the SDU 212 internally carries a solenoid 298 that is configured to interface with the interior of the vacuum canister 218 (e.g., via the suction tubing 214 or additional tubing) (see FIG. 6B). The solenoid can vent the negative pressure inside the canister by opening a valve 299 coupled to the solenoid (mechanically or electromagnetically) that opens the interior of the canister 218 to ambient pressure (see FIG. 6B). The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior of the canister 218. The venting may also be able to remove air bubbles inside the other lumens of the catheter and tubing sets. In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

In another configuration, a vacuum regulator 267 is disposed between the vacuum pump 266 and the canister 218, optionally in-line between the canister 218 and the SDU 212, to adjust or reduce the vacuum level generated by the vacuum pump 266 (see FIG. 6B) (a tank or accumulator can optionally be included along with the vacuum regulator 267 between the vacuum pump 266 and the canister 218). The vacuum regulator 267 can comprise an electro-pneumatic vacuum regulator, electronic vacuum regulator, or other type. For instance, while the vacuum pump 266 can generate in excess of −29.5 inHg (expressed as gauge pressure readings relative to atmospheric pressure (not absolute values)) vacuum at sea level and −24.5 inHg (expressed as gauge pressure readings relative to atmospheric pressure (not absolute values)) at about 5280 feet elevation, for certain procedures, such as in the pulmonary anatomy, it may be beneficial to have the SDU 212 generate a different vacuum level, such as approximately −18 inHg in one situation. To reduce the vacuum level, the vacuum regulator 267 can be incorporated into the system to control and stabilize the vacuum supplied to the canister 218 and optionally accommodate for variations in elevation where the system is being operated. The vacuum regulator 267 can be a manually-adjustable unit that uses a spring force balanced against an internal diaphragm valve to compensate for fluctuations in downstream flow. The diaphragm has atmospheric pressure on one side and the regulated vacuum on the other side, resulting in the regulated vacuum level is compensated for changes in elevation (i.e., the canister vacuum, if set to −20 inHg at sea level, would still contain −20 inHg at 5280 feet elevation). More generally, the vacuum regulator 267 allows adjustment of a canister vacuum level, as measured by a canister vacuum sensor 219 that communicates with the controller 235, from the maximum attainable (described above) down to zero (atmospheric pressure).

The vacuum regulator 267 can be adjusted to a nominal −18 inHg setpoint during the manufacturing process, after which the setpoint can be mechanically locked, such as by a fastener, cable tie, etc., or locked using other techniques, to prevent inadvertent change or adjustment. The vacuum regulator 267 can be installed internally within the SDU 212, with the SDU case 284 preventing unauthorized access to the vacuum regulator 267 using tamper-evident seals or other security mechanisms or structures. Alternatively, the vacuum regulator 267 can be disposed externally to the SDU case 284 and can optionally remain unlocked.

With continued reference to use of the system 200, a user connects a first connector 270 of the sterile suction tubing 216 to an aspiration luer 271 of the aspiration catheter 202 (similar to luer 115), and connects the second connector 272 of the sterile suction tubing 216 to port 274 in the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 278 is snapped in place, keeping the internal contents (e.g., saline) sterile. Systems configured for performing this type of sterile injection of high-pressure saline are described in U.S. Pat. No. 9,883,877, issued Feb. 6. 2018, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 may be abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 121 may couple to the saddle 283 by clipping, friction fitting, vacuum fitting, or other means.

The controller 235 operates the motor 287 to control movement of the saddle 283 and so move the piston 122 (FIG. 2) within the cassette 278 to pressurize fluid and deliver it to the aspiration catheter 202. The jet pressure from the opening 194 (FIG. 3) is proportional to the speed at which the motor 287 is driven and in one configuration the controller 235 can operate the motor 287 to operate in a range from about 280 rotations per minute (RPM) to about 340 RPM, resulting an jet pressures ranging from about 410 pounds per square inch (PSI) to about 707 PSI. Depending upon the particular implementation in which the system 200 is used, the motor 287 can be operated at different speeds. For instance, for pulmonary anatomy, a desired jet pressure can be achieved by running the motor 287 at a reduced speed of 310 RPM. The controller 235 can be operated through the one or more switches 297 to vary a speed of the motor 287 based upon the particular patient anatomy, such as varying the speed of the motor 287 between about 310 RPM to about 340 RPM, from about 280 RPM to about 410 RPM, about 280 RPM, 290 RPM, 300 RPM, 310 RPM, 320 RPM, 330 RPM, 340 RPM, 350 RPM, 360 RPM, 370 RPM, 380 RPM, 390 RPM, 400 RPM, 410 RPM, or within a range between any of the proceeding. Additionally, or as an alternate to the switches, the controller 235 can adjust the motor speed using a proportional-integral-derivative (PID) speed control algorithm or feedback loop to monitor and correct a speed of the motor 287 for any changes in load conditions.

In addition to the above, the controller 235 more generally control the operation and functionality of the SDU 212 and the system 200 as a whole. An operation of the vacuum pump 266, such as operating speed, etc., can be controlled by the controller 235 and associated or operatively connected hardware, firmware, etc. While reference is made to noise from the motor 287 controlling the saddle 283 is abated by internal foam sections 288, 289, where a lower audible noise of the vacuum pump 266 might be more desirable for a user, the controller 235 can vary the operating speed of the vacuum pump 266 to reduce the audible noise of the system 200, and more particularly noise from the vacuum pump 266. For instance, the controller 235 can operate the vacuum pump 266 at a reduced speed, such as approximately 60% of maximum speed, during start-up and then reduce the speed to about 30% of maximum when a desired vacuum is achieved, such as −27.5 inHg. The controller 235, and associated printed circuit board and other hardware, firmware, etc., controls the vacuum pump speed at a fixed setpoint. A voltage divider circuit on the board produces a speed input signal to the vacuum pump 266 (such as a vacuum pump motor controller), which sets the pump speed based upon the speed input signal, such as to approximately 60% of maximum in this particular configuration. Other start-up speeds can be achieved with other speed input signals.

In still another configuration, control of the vacuum pump 266 can be achieved through the one or more switches 297 in combination with the controller 235. For instance, one of the switches 297 is a manually operated potentiometer that can vary the speed of the vacuum pump 266 from about 0% to about 100% of maximum speed. The speed setting from the potentiometer can be monitored to measure the feedback voltage, and optionally present speed and potentiometer information to the user through the display 238.

Additionally, the controller 235 controls the information presented on the display 238 or through the SDU 212, such as alarms, warnings, pressure and flow information, or any other information, warnings, etc. related to the operation of the system 200. For instance, the controller 235 can include hardware, firmware, etc. that provides through the display, etc. notification of various alarms or other information, such as (i) a “No Suction” alarm notifying a user of vacuum leaks in the system, (ii) a “Terminal Vacuum Fault” alarm indicating a vacuum level that is too high or different from a predetermined threshold which occurs when the vacuum regulator 267 has failed, such as when the canister vacuum is lower than −20 inHg, lower than −18 inHg or some other predetermined threshold, (iii) “Terminal Motor Fault” alarm indicating a problem with operation of the cassette 278, and (iv) a splash screen, which displays for a few seconds upon power-up of the system 200, providing version information or other relevant information related to any of the hardware, firmware, etc. of the controller 235 or another component of the SDU 212.

### Signal-Based Clot Engagement Detection in Thrombectomy Devices

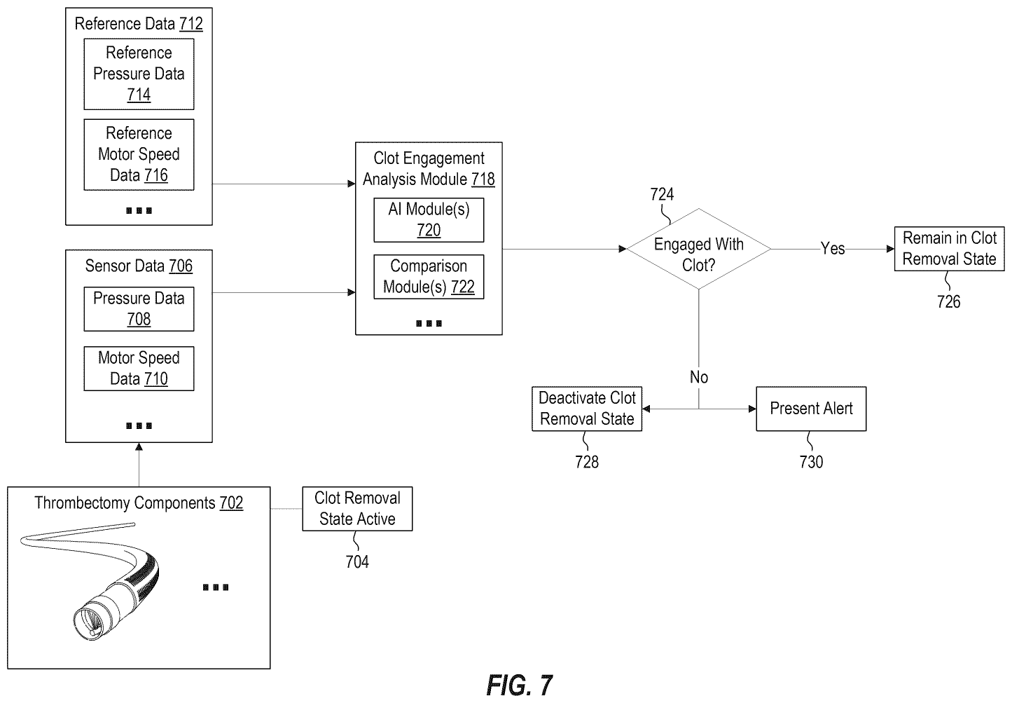

FIG. 7 illustrates a conceptual representation of determining clot engagement status during a clot removal state of a thrombectomy system based on sensor data, according to implementations of the present disclosure. In particular, FIG. 7 conceptually depicts thrombectomy components 702, which may include components of a thrombectomy system 100 as described hereinabove. For instance, the thrombectomy components 702 may comprise aspiration components such as an aspiration catheter and lumen, suction tubing, supply tubing (e.g., for jetting fluid), a vacuum pump/motor, a vacuum canister, a guidewire, a saline drive unit (SDU), and/or others (e.g., as indicated by the ellipsis within the thrombectomy components 702 of FIG. 7).

FIG. 7 depicts an instance where, at least initially, a clot removal state is active (as indicated by reference numeral 704). The clot removal state involves operation of the thrombectomy components 702 to facilitate clot removal. For instance, the vacuum motor/pump of the thrombectomy components 702 may generate negative pressure or aspiration pressure so as to aspirate thrombotic or embolic material (e.g., clot material) positioned at the distal end of the aspiration catheter. The thrombotic or embolic material may be macerated via a high-pressure saline jet formed via the SDU and supply tubing.

FIG. 7 furthermore depicts sensor data 706 associated with at least some of the thrombectomy components 702. The sensor data 706 may be monitored by a system (e.g., one or more control/processing circuitries of a thrombectomy system, or one or more devices in communication therewith) when the clot removal state is determined to be active. In the example of FIG. 7, the sensor data 706 includes pressure data 708, motor speed data 710, and/or others (indicated by the ellipsis within the sensor data 706). The pressure data 708 may be associated with the aspiration catheter/lumen, suction tubing, the vacuum canister, and/or other thrombectomy components 702 that may be pressurized or experience fluid flow. The pressure data 708 may comprise pressure values, other pressure metrics such as change in pressure over time, derivatives thereof, combinations thereof, etc. The motor speed data 710 may be associated with the vacuum pump/motor and/or other components usable to generate negative pressure in regions of the thrombectomy components 702. The motor speed data 710 may comprise motor speed values, change in motor speed values over time, derivatives thereof, combinations thereof, etc.

FIG. 7 conceptually represents at least part of the sensor data 706 being utilized as input to a clot engagement analysis module 718 (as indicated by the arrow extending from the sensor data 706 to the clot engagement analysis module 718). The clot engagement analysis module 718 may process the sensor data 706 (which is temporally subsequent to activation of the clot removal state) to determine whether the sensor data 706 indicates that the thrombectomy components 702 are engaged with clot material.

The clot engagement analysis module 718 may comprise any combination of software or hardware objects and may determine clot engagement status based on sensor data in various ways. In some instances, the clot engagement analysis module 718 is configured to compare the input sensor data 706 to reference data 712 associated with clot engagement or clot removal. By way of illustrative example, the clot engagement analysis module 718 may access or receive reference pressure data 714 for comparison to the pressure data 708 to determine whether the pressure data 708 indicates clot engagement (indicated in FIG. 7 by the arrow extending from the reference data 712 to the clot engagement analysis module 718).

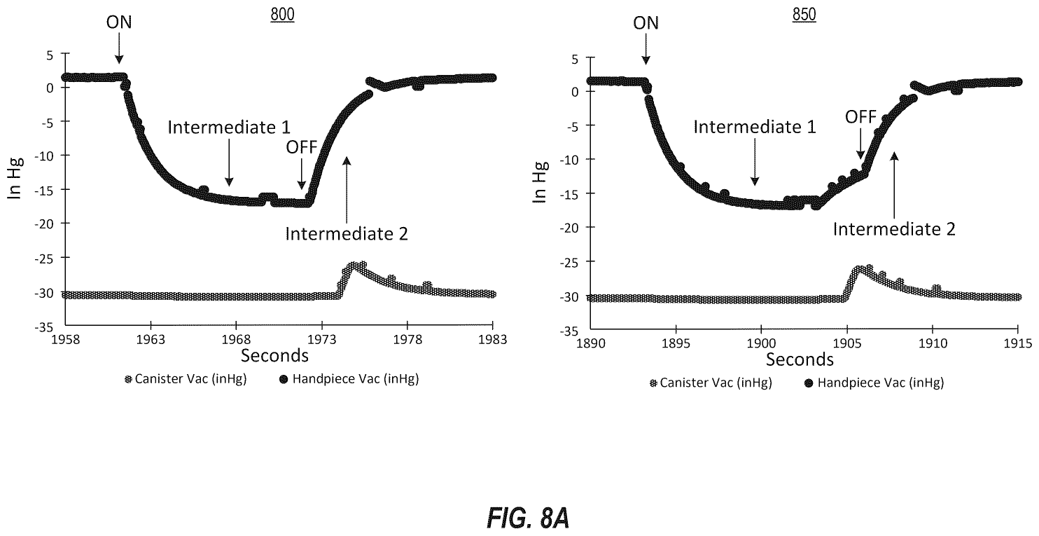

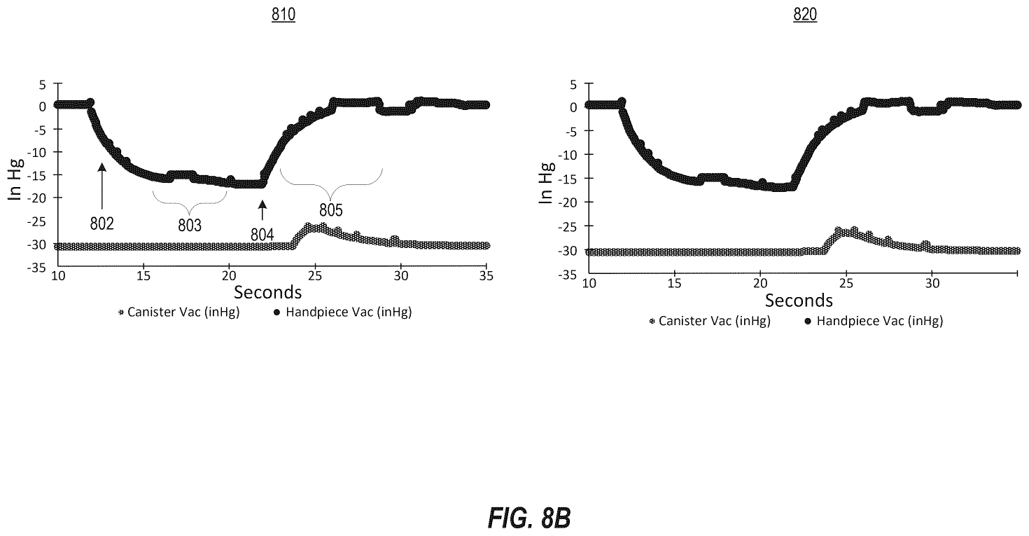

FIG. 8A provides graphs 800 and 850 depicting example pressure data over time. The pressure values of graphs 800 and 850 are representative of pressure measured by pressure sensors associated with an aspiration catheter/lumen or suction tubing of a thrombectomy system (referred to in FIG. 8A as “Handpiece Vac”) or with a vacuum canister of a thrombectomy system (referred to in FIG. 8A as a “Canister Vac”). The pressure values of graphs 800 and 850 represent different operational states of a thrombectomy system. For instance, graphs 800 and 850 depict a clot removal state start and a clot removal state end. The clot removal state start is indicated in graphs 800 and 850 by an “ON” label, whereas the clot removal state end is indicated by an “OFF” label. Graphs 800 and 850 include pressure values that precede the start of the clot removal state, that occur during operation of the clot removal state (indicated with an “Intermediate 1” label), and that follow ending of the clot removal state (e.g., a recovery phase indicated with an “Intermediate 2” label). Graph 800 shows pressure data associated with aspiration of blood during operation of a clot removal state, whereas graph 850 shows pressure data associated with aspiration of a clot during operation of a clot removal state. The pressure data in graphs 800 and 850 were captured in vivo over a 10-12 second pulse of clot removal state operation. FIG. 8B shows graphs 810 and 820, which illustrate additional pressure data (handpiece and canister pressure data) captured by two different catheters during operation of a clot removal state in the absence of clot material.

Continuing with the illustrative example, the pressure data of graph 800 may represent reference pressure data 714, which may be used by the clot engagement analysis module 718 to analyze input pressure data 708 to determine whether the thrombectomy components 702 with an active clot removal state are engaged with clot material. For instance, a clot engagement analysis module 718 may utilize comparison module(s) 722 to determine similarity (e.g., a similarity score) between (i) the pressure data 708 (e.g., pressure values, change in pressure values, or other values based thereon) that follow activation of the clot removal state in the thrombectomy components 702 and (ii) the reference pressure data 714 (e.g., reference pressure values, reference change in pressure values, or other values based thereon) that follow activation of the clot removal state. The pressure data 708 and reference pressure data 714 selected for comparison may be based on one or more time thresholds (e.g., a set amount of time following activation of the clot removal state). For instance, the pressure data 708 at or immediately following activation of the clot removal state may be compared to reference pressure data 714 for a corresponding time period relative to activation of the clot removal state (e.g., corresponding to the “ON” label in FIG. 8A or label 802 in FIG. 8B), or the pressure data 708 within a time window following activation of the clot removal state may be compared to reference pressure data 714 for a corresponding time period relative to activation of the clot removal state (e.g., corresponding to the “Intermediate 1” label in FIG. 8A or label 803 in FIG. 8B), or the pressure data 708 at or immediately following deactivation of the clot removal state may be compared to reference pressure data 714 for a corresponding time period relative to deactivation of the clot removal state (e.g., corresponding to the “OFF” label in FIG. 8A or label 804 in FIG. 8B), or the pressure data within a time window following deactivation of the clot removal state may be compared to reference pressure data for a corresponding time period relative to deactivation of the clot removal state (e.g., corresponding to the “Intermediate 2” label in FIG. 8A or label 805 in FIG. 8B).

Similarity may be determined in any suitable manner, such as, by way of non-limiting example, correlation coefficients, Euclidean distance (or other distance metrics), dynamic time warping, kernel methods, curve fitting, resampling, cross-correlation, statistical tests (e.g., means, variances, skewness, etc.), wavelet transform, time series analysis, statistical pattern recognition, combinations thereof, and/or others. In some implementations, similarity is determined based on extraction and analysis/comparison of key features/metrics of the input pressure data 708 and the reference pressure data 714, such as peaks (e.g., maxima), valleys (e.g., minima), slopes, inflection points, and/or other distinctive characteristics.

Output of the clot engagement analysis module 718 may indicate whether the aspiration components 702 are engaged with a clot or not after activation of the clot removal state (e.g., based on the similarity between the input pressure data 708 and the reference pressure data 714 satisfying one or more threshold similarity values or ranges). A thrombectomy system may implement different actions based on whether the clot engagement analysis module 718 determines, based on the sensor data 706 (and reference data 712), that the thrombectomy components 702 have encountered clot material. In some instances, the reference data 712 comprises different sets of data associated with engagement with different types of clots (e.g., clots with different composition, texture, elasticity, fragility, hardness/stiffness, texture, adherence, shape, etc.) or engagement status (e.g., clot engagement, clot non-engagement). In some implementations, based on comparing the sensor data 706 to the reference data 712 (or otherwise processing the sensor data 706), the clot engagement analysis module 718 outputs a label indicating the clot engagement status (e.g., engaged or not engaged) and/or the estimated type of clot the aspiration catheter is engaged with. In some embodiments, the label indicating the type of clot the aspiration catheter is engaged with may influence the type of aspiration state implemented by the thrombectomy system.

FIG. 7 conceptually depicts processing of the output of the clot engagement analysis module 718 via decision block 724, which indicates different actions that may be implemented based on whether the output of the clot engagement analysis module 718 indicates clot engagement (i.e., “Yes” extending from decision block 724) or clot non-engagement (i.e., “No” extending from decision block 724). In some implementations, in response to determining (via the clot engagement analysis module 718) that the thrombectomy components are engaged with a clot, the thrombectomy system may selectively remain in the clot removal state (indicated in FIG. 7 by reference numeral 726).

In some implementations, in response to determining (via the clot engagement analysis module 718) that the thrombectomy components 702 are not engaged with a clot, the thrombectomy system may selectively deactivate the clot removal state (indicated in FIG. 7 by reference numeral 728) and/or present an alert (indicated in FIG. 7 by reference numeral 730). Deactivating the clot removal state may comprise changing the operational settings of one or more of the thrombectomy components 702 (e.g., the vacuum pump/motor, suction tubing and/or aspiration catheter/lumen paths/configurations, SDU and/or supply line configurations, etc.). For instance, deactivating the clot removal state may comprise turning off the vacuum pump/motor, at least partially blocking or redirecting suction lines/tubing, etc. An alert may take on various forms, such as an audible alert, a visible alert, and/or a tactile alert (e.g., mechanical actuation of a foot pedal or other control).

Such functionality may advantageously enable thrombectomy systems to automatically determine, upon or soon after activation of a clot removal state, whether the thrombectomy components (e.g., an aspiration catheter) are engaged with a clot and automatically implement corrective action if the thrombectomy components are not engaged with a clot (e.g., deactivating aspiration). Systems that implement such techniques may advantageously mitigate patient blood loss, vessel wall damage, and/or other negative outcomes for patients.

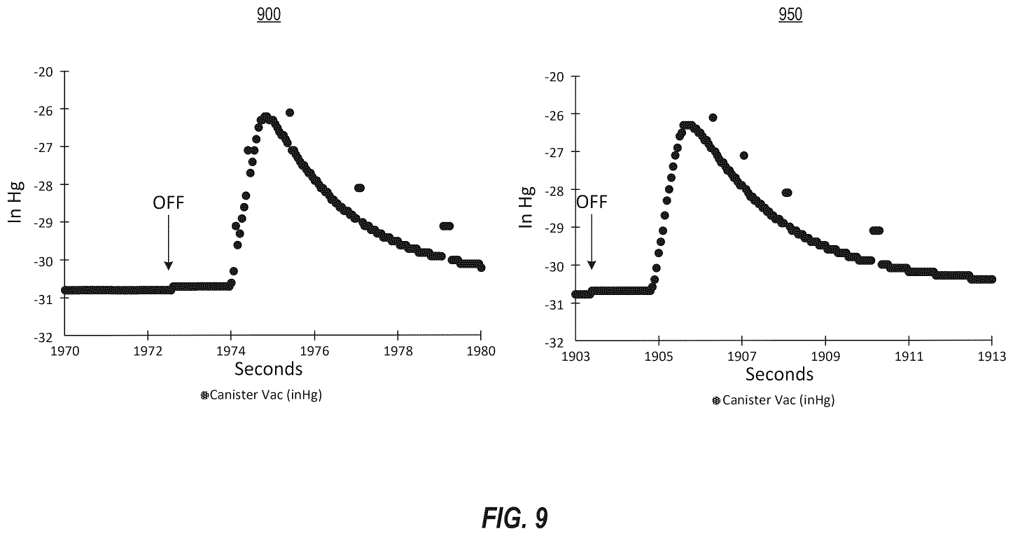

Pressure data 708 and reference pressure data 714 associated with any thrombectomy components may be utilized to determine clot engagement status, such as handpiece vacuum pressure data or canister vacuum pressure data. FIG. 9 provides graphs 900 and 950 representing reference pressure data 714 associated with a vacuum canister of a thrombectomy system (referred to as “Canister Vac” in FIG. 9) during clot removal state activation and deactivation and during aspiration of blood (graph 900) and aspiration of a clot (graph 950). Such reference pressure data 714 may be compared (via comparison module(s) 722) to pressure data 708 associated with a vacuum canister currently undergoing a current clot removal state to determine whether clot engagement is present in the current instance.

As another example, a clot engagement analysis module 718 may compare other types of data to determine clot engagement status, such as motor speed data 710 and reference motor speed data 716, and/or others such as flow rate or change in flow rate data (indicated by the ellipses in the sensor data 706 and reference data 712 of FIG. 7). Furthermore, in some instances, the clot engagement analysis module 718 utilizes AI module(s) 720 to determine similarity as discussed above and/or to infer or label clot engagement status based on sensor data 706. In some implementations, the AI module(s) 720 are trained on training data comprising sensor data (e.g., pressure, motor speed, flow rate, or other data captured during clot engagement with a clot removal state active) with corresponding ground truth labels indicating clot engagement status, thereby enabling the AI module(s) to output clot engagement labels in response to runtime sensor data.

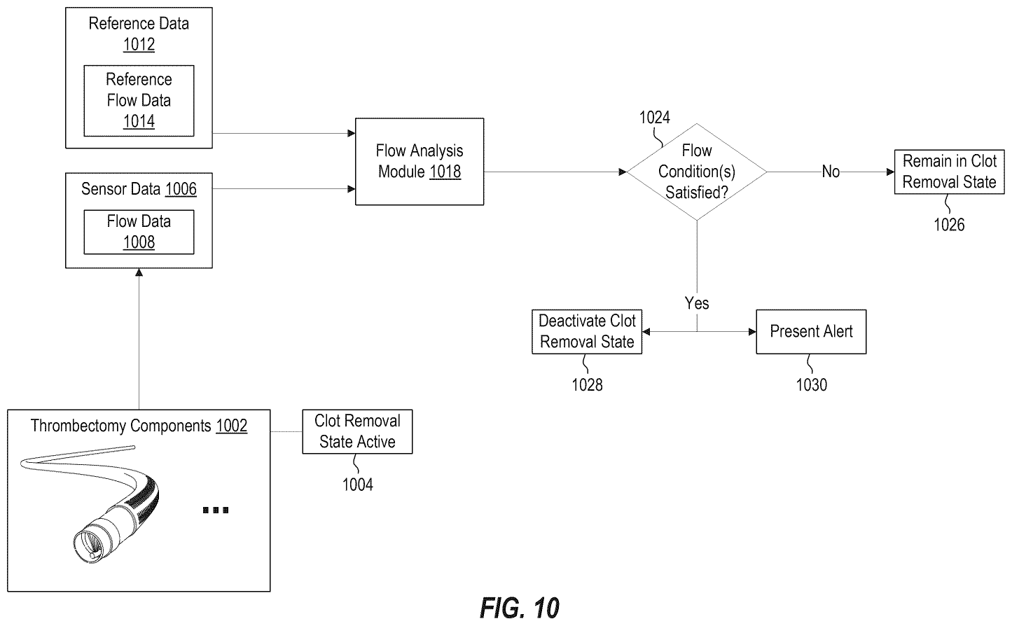

FIG. 10 illustrates a conceptual representation of controlling a clot removal state based on flow data, in accordance with implementations of the present disclosure. FIG. 10 conceptually depicts thrombectomy components 1002 (generally corresponding to thrombectomy components 702) with a clot removal state active (indicated by reference numeral 1004) and the acquisition of sensor data 1006 associated with at least some thrombectomy components 1002 during operation of the clot removal state. In the example of FIG. 10, the sensor data 1006 comprises flow data 1008. Flow data 1008 may be obtained via one or more fluid flow sensors and/or pressure sensors (as described hereinabove with reference to the pressure sensor 230 of FIG. 5). The flow data 1008 may comprise or indicate flow rate, change in flow rate, volume/amount of fluid passage/aspiration, and/or other metrics.

FIG. 10 also depicts reference data 1012, which may comprise reference flow data 1014. The reference flow data 1014 may comprise or indicate reference flow rate, reference change in flow rate, reference volume/amount of fluid passage/aspiration, and/or other metrics. By way of illustrative example, the reference flow data 1014 may indicate a threshold value or range for the volume of fluid aspiration that is not to be exceeded during clot aspiration procedures to ensure patient safety. In some instances, the threshold value or range for the volume of fluid aspiration is (automatically) selected based on patient attributes (e.g., age, sex, height, weight, BMI, body composition, medical conditions, etc.), such as by accessing patient EMR data or by user input of a practitioner.

During operation of the clot removal state, a thrombectomy system may acquire flow data 1008 and process the flow data 1008 via a flow analysis module 1018, which may compare the flow data 1008 to reference flow data 1014 to determine whether the flow data 1008 indicates that the clot removal state should be deactivated to maintain patient safety. For instance, the flow data 1008 may approximate the volume of patient blood lost by aspiration through an aspiration catheter, and the flow analysis module 1018 may compare the volume lost to a threshold volume indicated by the reference flow data 1014. If the volume lost meets or exceeds the volume indicated by the reference flow data 1014 (e.g., “Yes” extending from decision block 1024 in FIG. 10), the thrombectomy system may selectively deactivate the clot removal state (indicated by reference numeral 1028 in FIG. 10) and/or present an alert (indicated by reference numeral 1030 in FIG. 10). If the volume lost fails to meet or exceed the volume indicated by the reference flow data 1014 (e.g., “No” extending from decision block 1024 in FIG. 10), the thrombectomy system may selectively remain in the clot removal state (indicated by reference numeral 1026 in FIG. 10). Deactivating the clot removal state may comprise changing the operational settings of one or more of the thrombectomy components 1002 (e.g., the vacuum pump/motor, suction tubing and/or aspiration catheter/lumen paths/configurations, SDU and/or supply line configurations, etc.). For instance, deactivating the clot removal state may comprise turning off the vacuum pump/motor, at least partially blocking or redirecting suction lines/tubing, etc. An alert may take on various forms, such as an audible alert, a visible alert, and/or a tactile alert (e.g., mechanical actuation of a foot pedal or other control).

Such functionality may advantageously enable thrombectomy systems to refrain from maintaining an aspirating or clot removal state when the risk of excessive patient blood loss becomes too high, resulting in improved safety in thrombectomy or other clot removal procedures.

Although the example discussed with reference to FIG. 10 focuses, in at least some respects, on utilizing flow data 1008 and reference flow data 1014 processed by a flow analysis module 1018 to mitigate or control for excessive patient blood loss, other types of data may be utilized to this end, such as a time of operation/aspiration in a clot removal state (e.g., when a time of operation exceeds a reference time of operation, the thrombectomy system may selectively disable the clot removal state and/or present an alert to maintain patient safety).

### Adaptive Aspiration Based on Clot Detection in Thrombectomy Devices

The examples, discussed hereinabove with reference to FIGS. 7-9 dealt with monitoring and processing sensor data associated with thrombectomy components during operation of a clot removal state to determine clot engagement status. Based on the clot engagement status, the thrombectomy system may selectively deactivate the clot removal state (e.g., when the clot engagement status indicates clot non-engagement) or selectively remain in the clot removal state (e.g., when the clot engagement status indicates clot engagement). Similar operations may be performed during other operational states of thrombectomy components/systems.

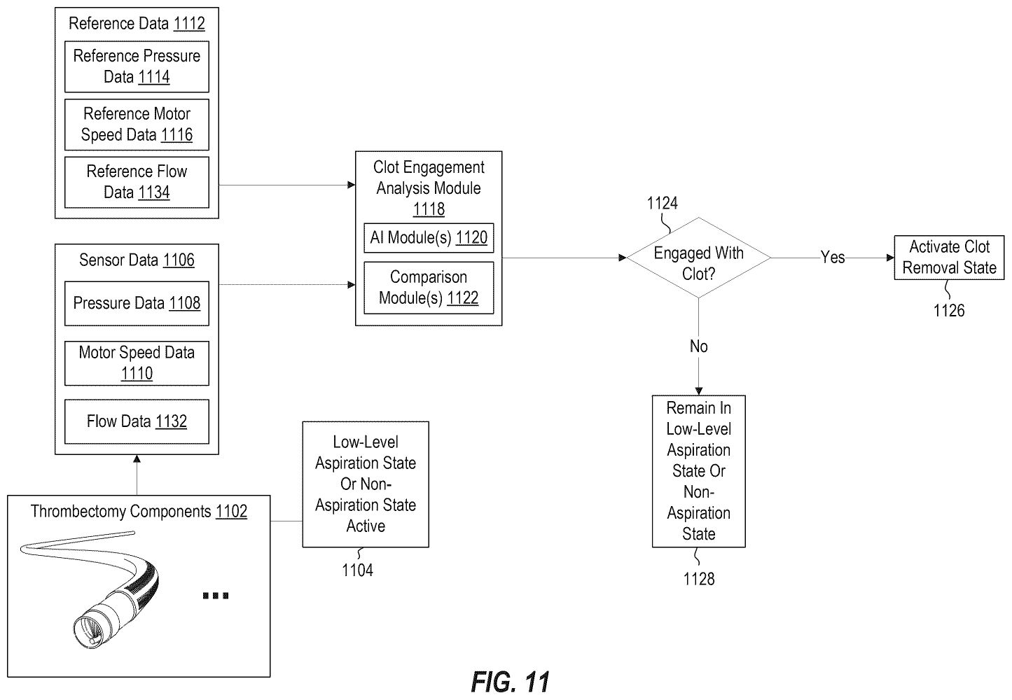

For example, FIG. 11 illustrates a conceptual representation of determining clot engagement status during a low-level aspiration state or a non-aspiration state of a thrombectomy system based on sensor data, according to implementations of the present disclosure. In particular, FIG. 11 conceptually depicts thrombectomy components 1102, which may include components of a thrombectomy system 100 as described hereinabove. For instance, the thrombectomy components 1102 may comprise aspiration components such as an aspiration catheter and lumen, suction tubing, supply tubing (e.g., for jetting fluid), a vacuum pump/motor, a vacuum canister, a guidewire, a saline drive unit (SDU), and/or others (e.g., as indicated by the ellipsis within the thrombectomy components 1102 of FIG. 11).

FIG. 11 depicts an instance where, at least initially, a low-level aspiration state or a non-aspiration state is active (as indicated by reference numeral 1104). The low-level aspiration state involves operation of the thrombectomy components 1102 at a lower setting or different parameters (e.g., relative to the clot removal state described hereinabove). For instance, the vacuum motor/pump of the thrombectomy components 702 operate at a low speed/RPM to generate negative pressure with low magnitude so as to facilitate weak aspiration through the aspiration catheter/lumen. In some instances, a user may activate the low-level aspiration state after navigation of the distal end of the aspiration catheter toward an approximate location of thrombotic or embolic material within patient vasculature. In some implementations, while the low-level aspiration state is active, the saline drive unit (e.g., SDU 212) of the thrombectomy components 1102 is deactivated or operates at a low level, such that a high-pressure saline jet is not present at the distal region of the catheter (e.g., aspiration catheter 202) during operation of the low-level aspiration state. Such functionality can mitigate vessel damage, prevent pushing of clot material distally away from the catheter (or into unintended vasculature), reduce blood cell lysis, and/or achieve other benefits.

The non-aspiration state comprises a state in which the vacuum motor/pump of the thrombectomy components 702 are deactivated or otherwise prevented from generating negative pressure through the aspiration catheter, such that no aspiration through the aspiration catheter/lumen is caused/performed. The non-aspiration state is a state in which the saline drive unit is deactivated such that a high-pressure saline jet is not present at the distal region of the aspiration catheter.

FIG. 11 furthermore depicts sensor data 1106 associated with at least some of the thrombectomy components 1102. The sensor data 1106 may be monitored by a system (e.g., one or more control/processing circuitries of a thrombectomy system, or one or more devices in communication therewith) when the low-level aspiration state or the non-aspiration state is determined to be active. In the example of FIG. 11, the sensor data 1106 includes pressure data 1108, motor speed data 1110, flow data 1132 and/or others (indicated by the ellipsis within the sensor data 706). The pressure data 1108 may be associated with the aspiration catheter/lumen, suction tubing, the vacuum canister, and/or other thrombectomy components 1102. The pressure data 1108 can be associated with aspiration components that may be pressurized or experience weak fluid flow during operation of the low-level aspiration state. The pressure data 1108 may comprise pressure values, pressure waves, other pressure metrics such as change in pressure over time, derivatives thereof, combinations thereof, etc. The motor speed data 1110 may be associated with the vacuum pump/motor and/or other components usable to generate weak negative pressure in regions of the thrombectomy components 1102. The motor speed data 1110 may comprise motor speed values, change in motor speed values over time, derivatives thereof, combinations thereof, etc. The flow data 1132 may comprise or indicate flow rate, change in flow rate, volume/amount of fluid passage/aspiration, and/or other metrics. Flow data 1132 may be obtained via one or more fluid flow sensors and/or pressure sensors (as described hereinabove with reference to the pressure sensor 230 of FIG. 5).

FIG. 11 conceptually represents at least part of the sensor data 1106 being utilized as input to a clot engagement analysis module 1118 (as indicated by the arrow extending from the sensor data 1106 to the clot engagement analysis module 1118). The clot engagement analysis module 1118 may process the sensor data 1106 (which is temporally subsequent to activation of the low-level aspiration state) to determine whether the sensor data 1106 indicates that the thrombectomy components 1102 are engaged with clot material.

The clot engagement analysis module 1118 may comprise any combination of software or hardware objects and may determine clot engagement status based on sensor data in various ways. In some instances, the clot engagement analysis module 1118 is configured to compare the input sensor data 1106 to reference data 1112 associated with clot engagement. By way of illustrative example, the clot engagement analysis module 1118 may access or receive reference pressure data 1114 for comparison to the pressure data 1108 to determine whether the pressure data 1108 indicates clot engagement (indicated in FIG. 11 by the arrow extending from the reference data 1112 to the clot engagement analysis module 1118).

The reference pressure data 1114 may conceptually correspond to the reference pressure data 714 described hereinabove with reference to FIGS. 7 and 8, although the reference pressure data 1114 may comprise or indicate pressure values, change in pressure values, pressure waves, changes in pressure waves, or derivatives thereof in association with aspiration components during operation of the low-level aspiration state (rather than the clot removal state, as discussed in association with reference pressure data 714).

In some instances, the pressure data 1108 indicates a blood pressure wave representative of the rhythmic oscillations in blood pressure that occur with each heartbeat of the subject of the clot removal operation. Such pressure data 1108 can be gathered via a pressure sensor associated with the aspiration catheter (e.g., enabling the aspiration catheter to essentially function as an invasive blood pressure monitor). Reference pressure data 1114 can include reference blood pressure wave data that captures normal blood pressure waves (e.g., before the aspiration catheter reaches a clot) and/or attenuated blood pressure waves (which attenuation can occur when the aspiration catheter is engaged with a clot).

The reference pressure data 1114 may be used by the clot engagement analysis module 1118 to analyze input pressure data 1108 to determine whether the thrombectomy components 1102 with an active low-level aspiration state or non-aspiration state are engaged with clot material. For instance, a clot engagement analysis module 1118 may utilize comparison module(s) 1122 to determine similarity (e.g., a similarity score) between (i) the pressure data 1108 (e.g., pressure values, change in pressure values, blood pressure waves, or other values based thereon) that follow implementation of the low-level aspiration state or the non-aspiration state in the thrombectomy components 1102 and (ii) the reference pressure data 1114 (e.g., reference pressure values, reference change in pressure values, reference blood pressure waves, or other values based thereon) that follow implementation of the low-level aspiration state or the non-aspiration state. The pressure data 1108 and reference pressure data 1114 selected for comparison may be based on one or more temporal thresholds (e.g., a set amount of time following implementation of the low-level aspiration state or the non-aspiration state).

Similarity may be determined in any suitable manner, such as, by way of non-limiting example, correlation coefficients, Euclidean distance (or other distance metrics), dynamic time warping, kernel methods, curve fitting, resampling, cross-correlation, statistical tests (e.g., means, variances, skewness, etc.), wavelet transform, time series analysis, statistical pattern recognition, combinations thereof, and/or others. In some implementations, similarity is determined based on extraction and analysis/comparison of key features/metrics of the input pressure data 1108 and the reference pressure data 1114, such as peaks (e.g., maxima), valleys (e.g., minima), slopes, inflection points, amplitudes (e.g., in the case of blood pressure waves) and/or other distinctive characteristics.

Output of the clot engagement analysis module 1118 may indicate whether the aspiration components 1102 are engaged with a clot or not after implementation of the low-level aspiration state or the non-aspiration state (e.g., based on the similarity between the input pressure data 1108 and the reference pressure data 1114 satisfying one or more threshold similarity values or ranges). A thrombectomy system may implement different actions based on whether the clot engagement analysis module 1118 determines, based on the sensor data 1106 (and reference data 1112), that the thrombectomy components 1102 have encountered clot material.

FIG. 11 conceptually depicts processing of the output of the clot engagement analysis module 1118 via decision block 1124, which indicates different actions that may be implemented based on whether the output of the clot engagement analysis module 1118 indicates clot engagement (i.e., “Yes” extending from decision block 1124) or clot non-engagement (i.e., “No” extending from decision block 1124). In some implementations, in response to determining (via the clot engagement analysis module 1118) that the thrombectomy components 1102 are not engaged with a clot, the thrombectomy system may selectively remain in the low-level aspiration state or the non-aspiration state (indicated in FIG. 11 by reference numeral 1128). Remaining in the low-level aspiration state or the non-aspiration state may enable users to continue to modify the positioning of the distal end of the aspiration catheter within patient vasculature in attempt to achieve clot engagement, while mitigating the possibility of vessel damage and/or other negative outcomes.

In some implementations, in response to determining (via the clot engagement analysis module 1118) that the thrombectomy components are engaged with a clot, the thrombectomy system may selectively activate the clot removal state (indicated in FIG. 11 by reference numeral 1126), such as by modifying operational parameters of the thrombectomy components 1102 (e.g., by increasing vacuum pump/motor speed, reconfiguring suction tubing/lining, etc.). In some instances, activation of the clot removal state includes causing the saline drive unit (e.g., SDU 212) of the thrombectomy components 1102 to deliver or increase delivery of saline to the distal region of the catheter (e.g., aspiration catheter 202) to cause a high-pressure saline jet to be present at the distal region of the catheter (e.g., released from orifice 194). Such functionality can enable maceration of clot material to improve clot removal speed and/or efficiency.

Once the clot removal state is active, a thrombectomy system may employ techniques similar to those discussed hereinabove with reference to FIGS. 7-10. For example, referring again to FIG. 7, where the clot removal state is active (indicated by reference numeral 704), the thrombectomy system may continue to collect sensor data 706 during clot aspiration. The clot engagement analysis module 718 may process the sensor data 706 to determine whether the sensor data 706 indicates completion of clot removal. For example, the comparison module(s) 722 may compare the sensor data 706 to reference data 712 associated with completion of clot removal (conceptually shown in FIG. 8A by the “OFF” or “Intermediate 2” labels) to determine whether the similarity between the sensor data 706 and the reference data 712 satisfies one or more similarity thresholds/conditions. If the similarity threshold(s)/condition(s) are satisfied (indicating that clot engagement is continuing), the thrombectomy system may selectively remain in the clot removal state (indicated in FIG. 7 by reference numeral 726). If the similarity threshold(s)/condition(s) are not satisfied (indicating that clot engagement has completed or that the clot has been removed), the thrombectomy system may selectively deactivate the clot removal state (indicated in FIG. 7 by reference numeral 728), return to the low-level aspiration state, and/or present an alert (indicated in FIG. 7 by reference numeral 730). In this regard, the clot engagement analysis module 718 may be characterized, in some instances, as a clot removal analysis module.

The functionality described hereinabove with reference to FIGS. 11 and 7 may assist practitioners in achieving clot engagement with reduced trial and error, which can result in shorter operation times, reduced patient blood loss, reduced damage to vessel walls, and/or other improvements to patient care.

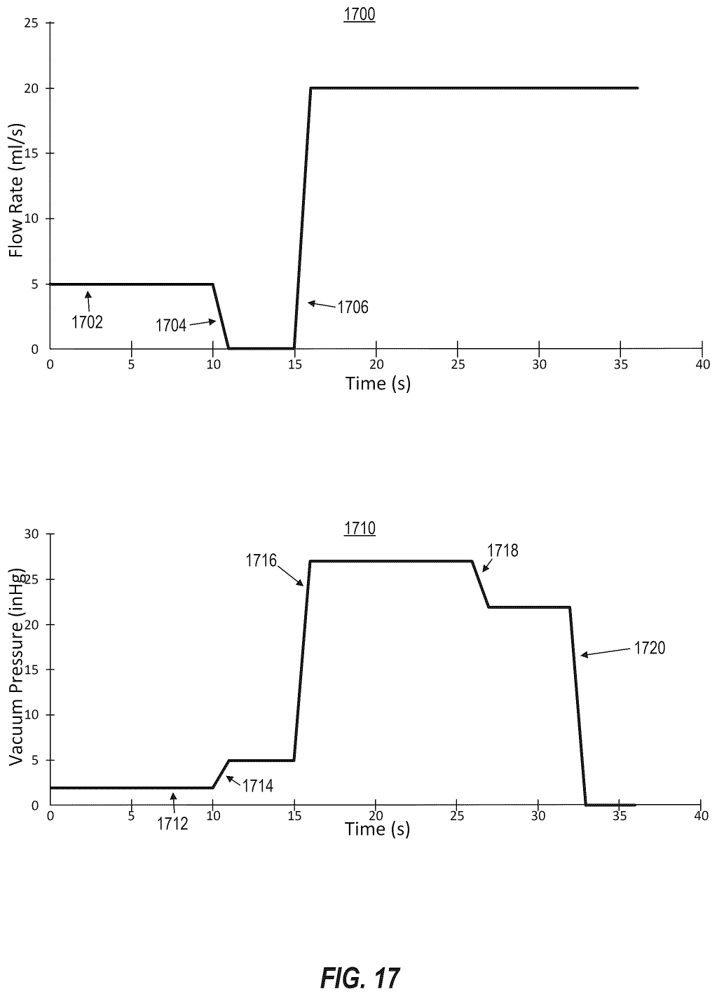

FIG. 17 depicts graph 1700, which provides a conceptual flow rate signal associated with operation of a thrombectomy system as described with reference to FIGS. 11 and 7. For instance, graph 1700 illustrates an initial flow rate 1702 during operation of a low-level aspiration state and shows a drop in flow rate 1704 resulting from engagement of the aspiration catheter with clot material. Graph 1700 also depicts an increase in flow rate 1706 resulting from activation of a clot removal state (e.g., the activation being based on detecting the drop in flow rate 1704 indicating clot engagement).

FIG. 17 also depicts graph 1710, which provides a conceptual pressure signal (e.g., handpiece vacuum pressure signal) associated with operation of a thrombectomy system as described with reference to FIGS. 11 and 7. For instance, graph 1710 illustrates an initial vacuum pressure 1712 during operation of a low-level aspiration state and shows an increase in vacuum pressure 1714 resulting from engagement of the aspiration catheter with clot material. Graph 1710 also depicts an increase in vacuum pressure 1716 resulting from activation of a clot removal state (e.g., the activation being based on detecting the increase in vacuum pressure 1714 indicating clot engagement). Graph 1710 further depicts a decrease in vacuum pressure 1718 resulting from completion of clot aspiration and shows a decrease in vacuum pressure 1720 resulting from deactivation of the clot removal state (e.g., the deactivation being based on detecting the decrease in vacuum pressure 1718 indicating clot removal completion).

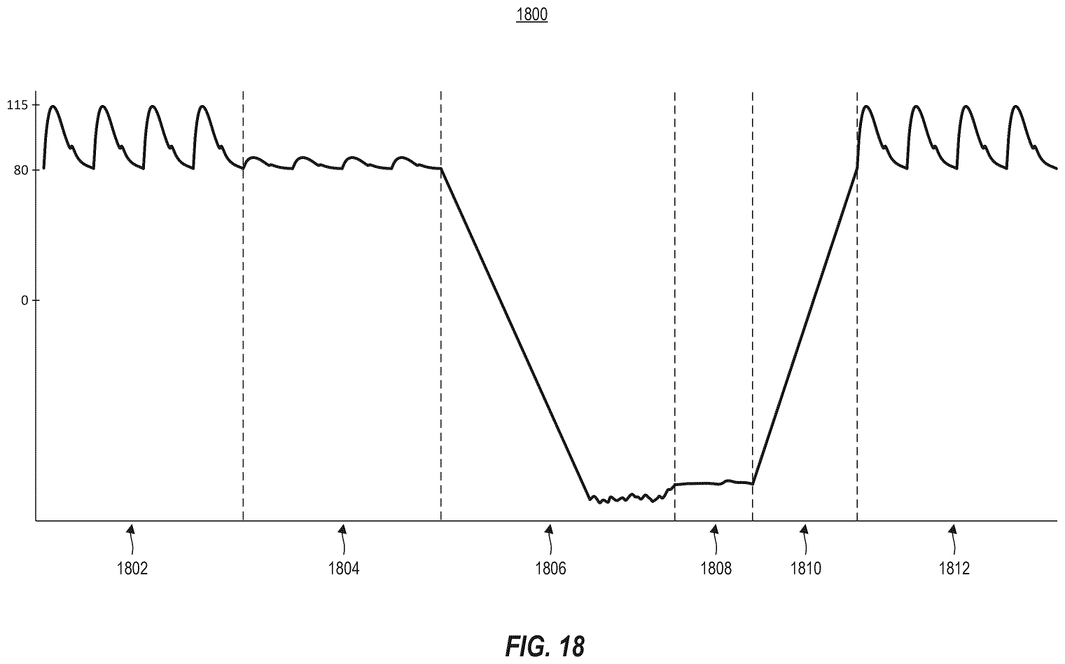

FIG. 18 depicts graph 1800, which provides a conceptual pressure signal (e.g., aspiration catheter pressure signal) associated with operation of a thrombectomy system as described with reference to FIGS. 11 and 7. Graph 1800 is separated into sections along the horizontal time axis (separated by vertical dashed lines and labeled with reference numerals). Graph 1800 illustrates initial pressure data in section 1802, capturing a blood pressure wave for a subject during implementation of a non-aspiration state for the aspiration catheter. The initial pressure data in section 1802 can be compared to reference pressure data (e.g., reference pressure data 1114), which can provide an indication that the aspiration catheter is not engaged with a clot. As the aspiration catheter is advanced within the body of the subject and engages with a clot (e.g., becoming at least partially plugged by the clot), the blood pressure wave becomes attenuated, as indicated by pressure data in section 1804. The pressure data in section 1804 can be compared to reference pressure data (e.g., reference pressure data 1114), which can provide an indication that the aspiration catheter is engaged with a clot, triggering activation of the clot removal state. Graph 1800 illustrates pressure data in section 1806 capturing a pressure drop associated with activation of the clot removal state. The pressure data in section 1806 can be compared to reference pressure data (e.g., reference pressure data 714), which can provide an indication that the aspiration catheter remains engaged with clot material throughout the aspiration of the clot, causing the thrombectomy system to remain in the clot removal state. Graph 1800 also shows pressure data in section 1808, capturing a pressure increase associated with completion of clot removal during operation of the clot removal state. The pressure data in section 1808 can be compared to reference pressure data (e.g., reference pressure data 714), which can provide an indication that clot removal is complete, triggering deactivation of the clot removal state. Graph 1800 also shows pressure data in section 1810, capturing a pressure increase associated with deactivation of the clot removal state. Graph 1800 also shows pressure data in section 1812, capturing the blood pressure wave for the subject as the thrombectomy system returns to the non-aspiration state.

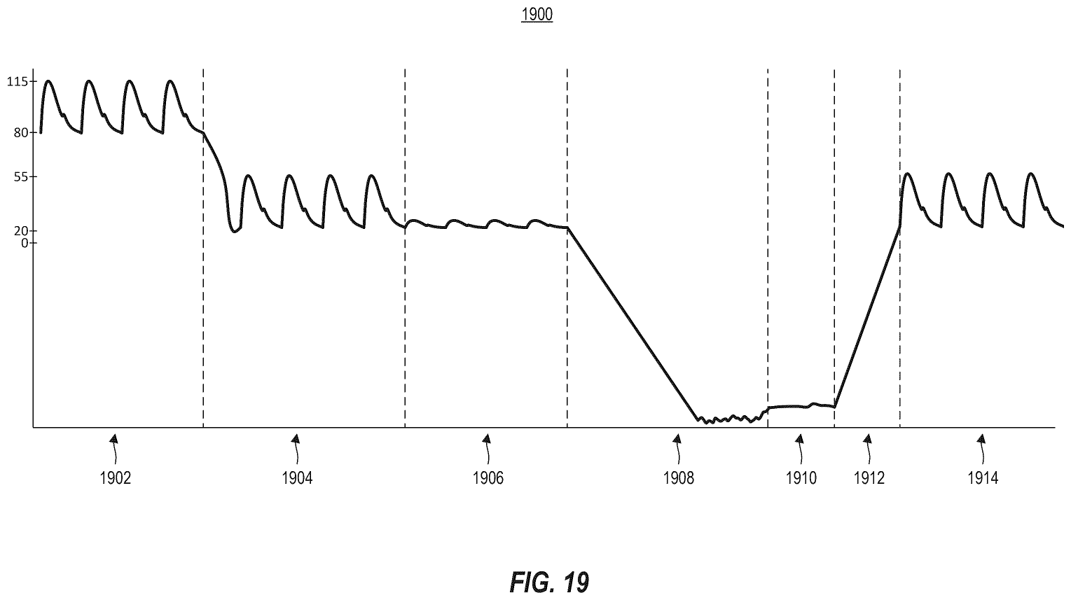

FIG. 19 depicts graph 1900, which provides a conceptual pressure signal (e.g., aspiration catheter pressure signal) associated with operation of a thrombectomy system as described with reference to FIGS. 11 and 7. Similar to graph 1800, graph 1900 is separated into sections along the horizontal time axis (separated by vertical dashed lines and labeled with reference numerals). Graph 1900 illustrates initial pressure data in section 1902, capturing a blood pressure wave for a subject during implementation of a non-aspiration state for the aspiration catheter. Graph 1900 also illustrates pressure data in section 1904 capturing the blood pressure wave for the subject during implementation of a low-level aspiration state for the aspiration catheter. The low-level aspiration state can be manually or automatically activated (e.g., based on estimated proximity to clot material). The pressure data in section 1904 can be compared to reference pressure data (e.g., reference pressure data 1114), which can provide an indication that the aspiration catheter is not engaged with a clot. As the aspiration catheter is advanced within the body of the subject and engages with a clot (e.g., becoming at least partially plugged by the clot), the blood pressure wave becomes attenuated, as indicated by pressure data in section 1906. The pressure data in section 1906 can be compared to reference pressure data (e.g., reference pressure data 1114), which can provide an indication that the aspiration catheter is engaged with a clot, triggering activation of the clot removal state. Graph 1900 illustrates pressure data in section 1908 capturing a pressure drop associated with activation of the clot removal state. The pressure data in section 1908 can be compared to reference pressure data (e.g., reference pressure data 714), which can provide an indication that the aspiration catheter remains engaged with clot material throughout the aspiration of the clot, causing the thrombectomy system to remain in the clot removal state. Graph 1900 also shows pressure data in section 1910 capturing a pressure increase associated with completion of clot removal during operation of the clot removal state. The pressure data in section 1910 can be compared to reference pressure data (e.g., reference pressure data 714), which can provide an indication that clot removal is complete, triggering deactivation of the clot removal state. Graph 1900 also shows pressure data in section 1912, capturing a pressure increase associated with deactivation of the clot removal state. Graph 1900 also shows pressure data in section 1914, capturing the blood pressure wave for the subject as the thrombectomy system returns to the low-level aspiration state (alternatively, the thrombectomy system can return to the non-aspiration state).

Following a procedure similar to those outlined in graphs 1800 and 1900 can contribute to increased engagement with thrombus, resulting in reduced aspiration time and/or blood loss for thrombectomy operations.

Although examples discussed above with reference to FIG. 11 are focused, in at least some respects, on utilizing pressure data 1108 and reference pressure data 1114 to determine clot engagement status via comparison module(s) 1122 of a clot engagement analysis module 1118, other variations are within the scope of the present disclosure. For example, a clot engagement analysis module 1118 may compare other types of data to determine clot engagement status, such as motor speed data 1110 and reference motor speed data 1116, flow data 1132 and reference flow data 1134, and/or others (indicated by the ellipses in the sensor data 1106 and reference data 1112 of FIG. 11). Furthermore, in some instances, the clot engagement analysis module 1118 utilizes AI module(s) 1120 to determine similarity as discussed above and/or to infer or label clot engagement status based on sensor data 1106. In some implementations, the AI module(s) 1120 are trained on training data comprising sensor data (e.g., pressure, motor speed, flow rate, or other data captured during clot engagement with a low-level aspiration state active) with corresponding ground truth labels indicating clot engagement status, thereby enabling the AI module(s) to output clot engagement labels in response to runtime sensor data.

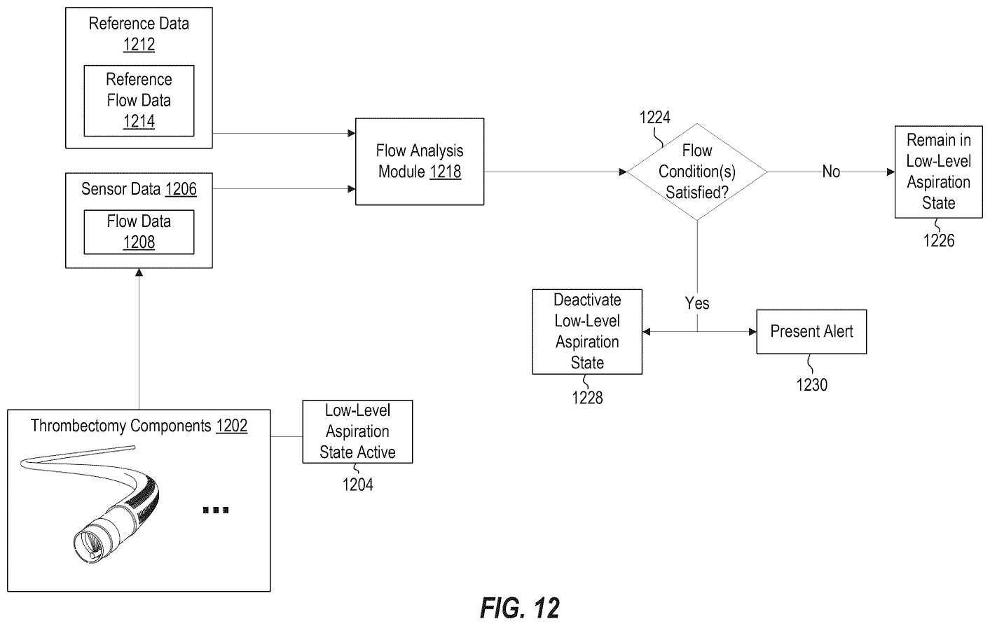

FIG. 12 illustrates a conceptual representation of controlling a low-level aspiration state based on flow data, in accordance with implementations of the present disclosure. FIG. 12 conceptually depicts thrombectomy components 1202 (generally corresponding to thrombectomy components 1102) with a low-level aspiration state active (indicated by reference numeral 1204) and the acquisition of sensor data 1206 associated with at least some thrombectomy components 1202 during operation of the low-level aspiration state. In the example of FIG. 12, the sensor data 1206 comprises flow data 1208. Flow data 1208 may be obtained via one or more fluid flow sensors and/or pressure sensors (as described hereinabove with reference to the pressure sensor 230 of FIG. 5). The flow data 1208 may comprise or indicate flow rate, change in flow rate, volume/amount of fluid passage/aspiration, and/or other metrics.

FIG. 12 also depicts reference data 1212, which may comprise reference flow data 1214. The reference flow data 1214 may comprise or indicate reference flow rate, reference change in flow rate, reference volume/amount of fluid passage/aspiration, and/or other metrics. By way of illustrative example, the reference flow data 1214 may indicate a threshold value or range for the volume of fluid aspiration that is not to be exceeded during clot aspiration procedures (whether low-level or not) to ensure patient safety. In some instances, the threshold value or range for the volume of fluid aspiration is (automatically) selected based on patient attributes (e.g., age, sex, height, weight, BMI, body composition, medical conditions, etc.), such as by accessing patient EMR data or by user input of a practitioner.

During operation of the low-level aspiration state, a thrombectomy system may acquire flow data 1208 and process the flow data 1208 via a flow analysis module 1218, which may compare the flow data 1208 to reference flow data 1214 to determine whether the flow data 1208 indicates that the low-level aspiration state should be deactivated to maintain patient safety. For instance, the flow data 1208 may approximate the volume of patient blood lost by aspiration through an aspiration catheter, and the flow analysis module 1218 may compare the volume lost to a threshold volume indicated by the reference flow data 1214. If the volume lost meets or exceeds the volume indicated by the reference flow data 1214 (e.g., “Yes” extending from decision block 1224 in FIG. 12), the thrombectomy system may selectively deactivate the low-level aspiration state (indicated by reference numeral 1228 in FIG. 12) and/or present an alert (indicated by reference numeral 1230 in FIG. 12). If the volume lost fails to meet or exceed the volume indicated by the reference flow data 1214 (e.g., “No” extending from decision block 1224 in FIG. 12), the thrombectomy system may selectively remain in the low-level aspiration state (indicated by reference numeral 1226 in FIG. 12). Deactivating the clot removal state may comprise changing the operational settings of one or more of the thrombectomy components 1202 (e.g., the vacuum pump/motor, suction tubing and/or aspiration catheter/lumen paths/configurations, SDU and/or supply line configurations, etc.). For instance, deactivating the low-level aspiration state may comprise turning off the vacuum pump/motor, at least partially blocking or redirecting suction lines/tubing, etc. An alert may take on various forms, such as an audible alert, a visible alert, and/or a tactile alert (e.g., mechanical actuation of a foot pedal or other control).

Such functionality may advantageously enable thrombectomy systems to refrain from maintaining a low-level aspiration or clot removal state when the risk of excessive patient blood loss becomes too high, resulting in improved safety in thrombectomy or other clot removal procedures.

Although the example discussed with reference to FIG. 12 focuses, in at least some respects, on utilizing flow data 1208 and reference flow data 1214 processed by a flow analysis module 1218 to mitigate or control for excessive patient blood loss, additional or alternative types of data may be utilized to this end, such as a time of operation/aspiration in a low-level aspiration state (e.g., when a time of operation exceeds a reference time of operation, the thrombectomy system may selectively disable the low-level aspiration state and/or present an alert to maintain patient safety). As another example, a thrombectomy system may directly measure volume, weight, or other characteristics of material aspirated through the aspiration catheter (e.g., material that reaches a collection region of the vacuum canister) to determine whether to selectively deactivate an aspiration state. For instance, the system may compare the weight or volume of aspirated material (e.g., subtracting saline introduced by the aspiration catheter) to one or more thresholds (represented in reference data) to determine whether to selectively deactivate an aspiration state.

### Example Methods

The following discussion now refers to a number of methods and method acts that may be performed in accordance with the present disclosure. Although the method acts are discussed in a certain order and illustrated in a flow chart as occurring in a particular order, no particular ordering is required unless specifically stated, or required because an act is dependent on another act being completed prior to the act being performed. One will appreciate that certain embodiments of the present disclosure may omit one or more of the acts described herein.

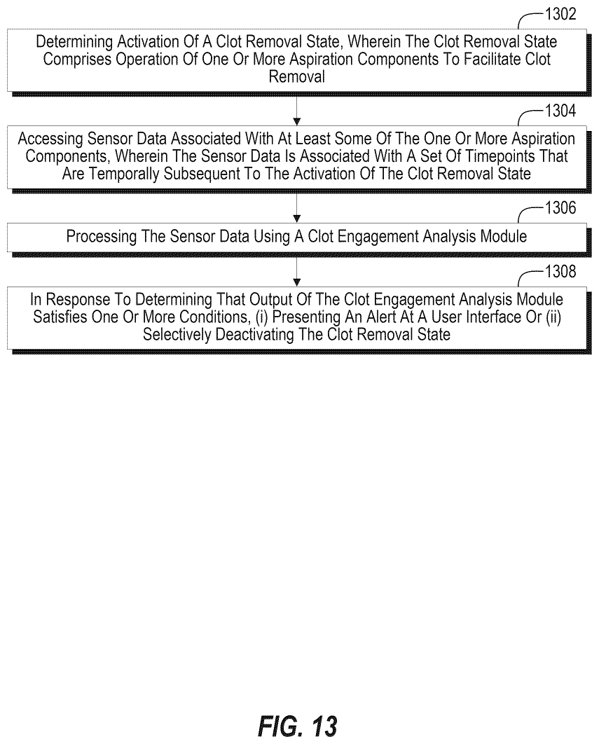

FIGS. 13-16 illustrate example flow diagrams 1300, 1400, 1500, and 1600, respectively, depicting acts associated with controlling operation of a thrombectomy system based on sensor data.

Act 1302 of flow diagram 1300 of FIG. 13 includes determining activation of a clot removal state, wherein the clot removal state comprises operation of one or more aspiration components to facilitate clot removal. In some instances, the one or more aspiration components comprise a vacuum pump, a vacuum canister, and suction tubing.

Act 1304 of flow diagram 1300 includes accessing sensor data associated with at least some of the one or more aspiration components, wherein the sensor data is associated with a set of timepoints that are temporally subsequent to the activation of the clot removal state. In some implementations, the sensor data comprises pressure data associated with the vacuum canister and/or the suction tubing. In some examples, the pressure data indicates a change in pressure over time. The clot engagement analysis module may be configured to compare the change in pressure over time to a reference change in pressure over time associated with clot engagement or clot removal.

Act 1306 of flow diagram 1300 includes processing the sensor data using a clot engagement analysis module. In some instances, the clot engagement analysis module is configured to compare the pressure data to reference pressure data associated with clot engagement or clot removal. In some implementations, the clot engagement analysis module comprises one or more AI modules trained on pressure data of vacuum canisters and/or suction tubing during clot engagement or clot removal. In some examples, the one or more AI modules are configured to utilize the pressure data as input and output a label indicating clot engagement status.

Act 1308 of flow diagram 1300 includes in response to determining that output of the clot engagement analysis module satisfies one or more conditions, (i) presenting an alert at a user interface or (ii) selectively deactivating the clot removal state. In some instances, selectively deactivating the clot removal state comprises selectively deactivating or modifying operation of the vacuum pump. In some implementations, the alert comprises an audible or visual alert.

In some examples, one or more additional actions may be performed in parallel with one or more of the acts of flow diagram 1300 discussed above, such as (i) during operation of the clot removal state, monitor (a) a duration of operation and/or (b) flow data obtained by a flow sensor associated with the thrombectomy system; and (ii) in response to determining that the duration of operation or the flow data satisfy one or more conditions, selectively deactivate the clot removal state.

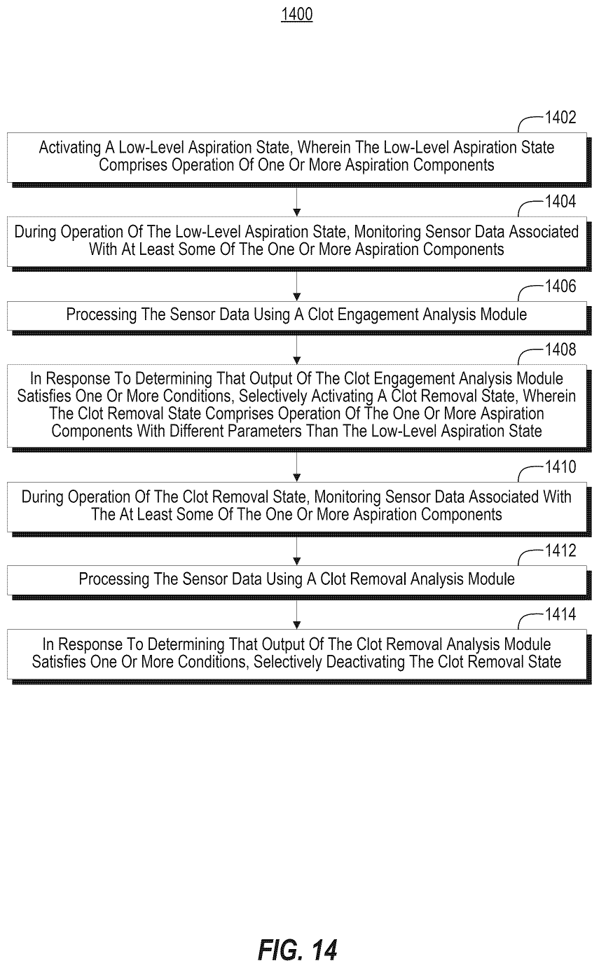

Act 1402 of flow diagram 1400 of FIG. 14 includes activating a low-level aspiration state, wherein the low-level aspiration state comprises operation of one or more aspiration components. In some instances, the one or more aspiration components comprise a vacuum pump, a vacuum canister, and suction tubing. In some instances, the one or more aspiration components comprise a saline drive unit. In some instances, the low-level aspiration state comprises refraining from activating a high-pressure saline jet at a distal region of an aspiration catheter.

Act 1404 of flow diagram 1400 includes during operation of the low-level aspiration state, monitoring sensor data associated with at least some of the one or more aspiration components. In some implementations, the sensor data comprises pressure data associated with the vacuum canister and/or the suction tubing. In some examples, the pressure data indicates a change in pressure over time. The clot engagement analysis module may be configured to compare the change in pressure over time to a reference change in pressure over time associated with clot engagement or clot removal.

Act 1406 of flow diagram 1400 includes processing the sensor data using a clot engagement analysis module. In some instances, the clot engagement analysis module is configured to compare the pressure data to reference pressure data associated with clot engagement or clot removal. In some implementations, the clot engagement analysis module comprises one or more AI modules trained on pressure data of vacuum canisters and/or suction tubing during clot engagement or clot removal. In some examples, the one or more AI modules are configured to utilize the pressure data as input and output a label indicating clot engagement status.

Act 1408 of flow diagram 1400 includes, in response to determining that output of the clot engagement analysis module satisfies one or more conditions, selectively activating a clot removal state, wherein the clot removal state comprises operation of the one or more aspiration components with different parameters than the low-level aspiration state. In some instances, activation of the clot removal state comprises increasing vacuum pressure at a distal region of an aspiration catheter for aspirating clot material. In some instances, activation of the clot removal state comprises activating a high-pressure saline jet at a distal region of an aspiration catheter for macerating clot material.

Act 1410 of flow diagram 1400 includes, during operation of the clot removal state, monitoring sensor data associated with the at least some of the one or more aspiration components.

Act 1412 of flow diagram 1400 includes processing the sensor data using a clot removal analysis module.

Act 1414 of flow diagram 1400 includes, in response to determining that output of the clot removal analysis module satisfies one or more conditions, selectively deactivating the clot removal state.

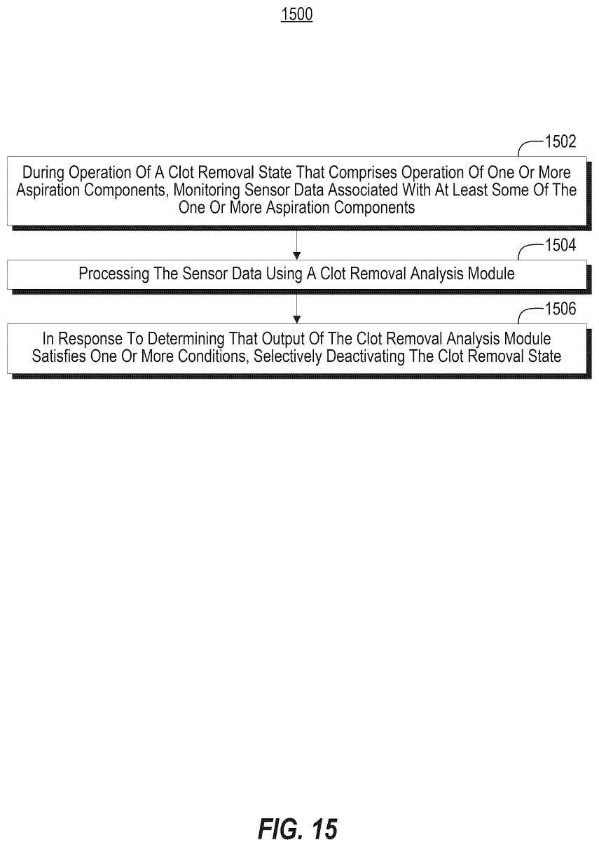

Act 1502 of flow diagram 1500 of FIG. 15 includes, during operation of a clot removal state that comprises operation of one or more aspiration components, monitoring sensor data associated with at least some of the one or more aspiration components. In some instances, the one or more aspiration components comprise a vacuum pump, a vacuum canister, and suction tubing. In some implementations, the sensor data comprises pressure data associated with the vacuum canister and/or the suction tubing. In some examples, the pressure data indicates a change in pressure over time. The clot removal analysis module may be configured to compare the change in pressure over time to a reference change in pressure over time associated with completion of clot removal.

Act 1504 of flow diagram 1500 includes processing the sensor data using a clot removal analysis module. In some instances, the clot removal analysis module is configured to compare the pressure data to reference pressure data associated with completion of clot removal. In some implementations, the clot removal analysis module comprises one or more AI modules trained on pressure data of vacuum canisters and/or suction tubing during completion of clot removal. In some examples, the one or more AI modules are configured to utilize the pressure data as input and output a label indicating clot removal completion status.

Act 1506 of flow diagram 1500 includes, in response to determining that output of the clot removal analysis module satisfies one or more conditions, selectively deactivating the clot removal state.

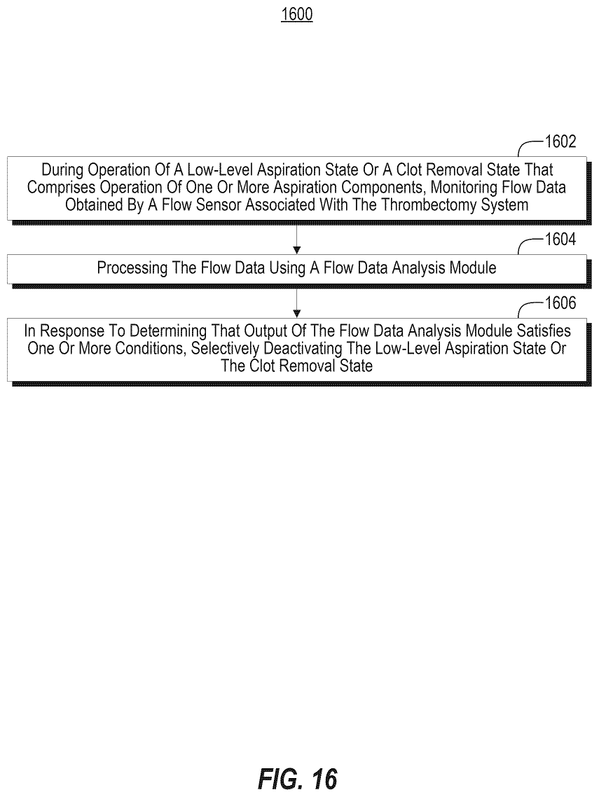

Act 1602 of flow diagram 1600 of FIG. 16 includes, during operation of a low-level aspiration state or a clot removal state that comprises operation of one or more aspiration components, monitoring flow data obtained by a flow sensor associated with the thrombectomy system. In some instances, the one or more aspiration components comprise a vacuum pump, a vacuum canister, and suction tubing. In some implementations, the flow sensor is connected to the suction tubing.

Act 1604 of flow diagram 1600 includes processing the flow data using a flow data analysis module.

Act 1606 of flow diagram 1600 includes, in response to determining that output of the flow data analysis module satisfies one or more conditions, selectively deactivating the low-level aspiration state or the clot removal state. In some examples, the output of the flow data analysis module indicates an amount of fluid aspirated via operation of the one or more aspiration components. In some instances, the one or more conditions comprise one or more threshold amounts of fluid.

### Example Embodiments

Embodiments disclosed herein can include those in the following numbered clauses:

Clause 1. A thrombectomy system, comprising: one or more processors; and one or more computer-readable recording media that store instructions that are executable by the one or more processors to configure the thrombectomy system to: activate a low-level aspiration state or a non-aspiration state, wherein the low-level aspiration state comprises operation of one or more aspiration components; during operation of the low-level aspiration state or the non-aspiration state, monitor sensor data associated with at least some of the one or more aspiration components; process the sensor data using a clot engagement analysis module; and after determining that output of the clot engagement analysis module satisfies one or more conditions, selectively activate a clot removal state, wherein the clot removal state comprises operation of the one or more aspiration components with different parameters than the low-level aspiration state.

Clause 2. The thrombectomy system of clause 1, wherein the one or more aspiration components comprise an aspiration catheter, a vacuum pump, a vacuum canister, and suction tubing.

Clause 3. The thrombectomy system of clause 1, wherein the one or more aspiration components comprises a saline drive unit.

Clause 4. The thrombectomy system of clause 1, wherein the low-level aspiration state comprises refraining from activating a high-pressure saline jet at a distal region of an aspiration catheter.

Clause 5. The thrombectomy system of clause 1, wherein activation of the clot removal state comprises increasing vacuum pressure at a distal region of an aspiration catheter.

Clause 6. The thrombectomy system of clause 1, wherein activation of the clot removal state comprises activating a high-pressure saline jet at a distal region of an aspiration catheter.

Clause 7. The thrombectomy system of clause 2, wherein the sensor data comprises pressure data associated with the aspiration catheter, the vacuum canister, and/or the suction tubing.

Clause 8. The thrombectomy system of clause 7, wherein the clot engagement analysis module is configured to compare the pressure data to reference pressure data associated with clot engagement or clot removal.

Clause 9. The thrombectomy system of clause 7, wherein the pressure data indicates a blood pressure wave, and wherein the clot engagement analysis module is configured to compare the blood pressure wave to a reference blood pressure wave associated with clot engagement.

Clause 10. The thrombectomy system of clause 7, wherein the pressure data indicates a change in pressure over time, and wherein the clot engagement analysis module is configured to compare the change in pressure over time to a reference change in pressure over time associated with clot engagement or clot removal.

Clause 11. The thrombectomy system of clause 7, wherein the clot engagement analysis module comprises one or more AI modules trained on pressure data of vacuum canisters and/or suction tubing during clot engagement or clot removal.

Clause 12. The thrombectomy system of clause 11, wherein the one or more AI modules are configured to utilize the pressure data as input and output a label indicating clot engagement status.

Clause 13. The thrombectomy system of clause 1, the instructions are executable by the one or more processors to further configure the thrombectomy system to: during operation of the clot removal state, monitor sensor data associated with the at least some of the one or more aspiration components; process the sensor data using a clot removal analysis module; and after determining that output of the clot removal analysis module satisfies one or more conditions, selectively deactivate the clot removal state.

Clause 14. A thrombectomy system, comprising: one or more processors; and one or more computer-readable recording media that store instructions that are executable by the one or more processors to configure the thrombectomy system to: during operation of a clot removal state that comprises operation of one or more aspiration components, monitor sensor data associated with at least some of the one or more aspiration components; process the sensor data using a clot removal analysis module; and after determining that output of the clot removal analysis module satisfies one or more conditions, selectively deactivate the clot removal state.

Clause 15. The thrombectomy system of clause 10, wherein the one or more aspiration components comprise a vacuum pump, a vacuum canister, and suction tubing.

Clause 16. The thrombectomy system of clause 15, wherein the sensor data comprises pressure data associated with the vacuum canister and/or the suction tubing.

Clause 17. The thrombectomy system of clause 16, wherein the clot removal analysis module is configured to compare the pressure data to reference pressure data associated with completion of clot removal.

Clause 18. The thrombectomy system of clause 16, wherein the pressure data indicates a change in pressure over time, and wherein the clot removal analysis module is configured to compare the change in pressure over time to a reference change in pressure over time associated with completion of clot removal.

Clause 19. The thrombectomy system of clause 16, wherein the clot removal analysis module comprises one or more AI modules trained on pressure data of vacuum canisters and/or suction tubing during completion of clot removal.

Clause 20. The thrombectomy system of clause 19, wherein the one or more AI modules are configured to utilize the pressure data as input and output a label indicating clot removal completion status.

Clause 21. A thrombectomy system, comprising: one or more processors; and one or more computer-readable recording media that store instructions that are executable by the one or more processors to configure the thrombectomy system to: during operation of a low-level aspiration state or a clot removal state that comprises operation of one or more aspiration components, monitor flow data obtained by a flow sensor associated with the thrombectomy system; process the flow data using a flow data analysis module; and after determining that output of the flow data analysis module satisfies one or more conditions, selectively deactivate the low-level aspiration state or the clot removal state.

Clause 22. The thrombectomy system of clause 21, wherein the one or more aspiration components comprise a vacuum pump, a vacuum canister, and suction tubing.

Clause 23. The thrombectomy system of clause 22, wherein the flow sensor is connected to the suction tubing.

Clause 24. The thrombectomy system of clause 21, wherein the output of the flow data analysis module indicates an amount of fluid aspirated via operation of the one or more aspiration components.

Clause 25. The thrombectomy system of clause 24, wherein the one or more conditions comprise one or more threshold amounts of fluid.

### Additional Details Related to Implementing the Disclosed Embodiments

The principles disclosed herein may be implemented in various formats. For example, at least some techniques discussed herein may be performed as a method that includes various acts for achieving particular results or benefits. In some instances, the techniques discussed herein are represented in computer-executable instructions that may be stored on one or more hardware storage devices. The computer-executable instructions may be executable by one or more processors to carry out (or to configure a system to carry out) the disclosed techniques. In some embodiments, a system may be configured to send the computer-executable instructions to a remote device to configure the remote device for carrying out the disclosed techniques.

Systems for implementing the disclosed embodiments may include various components, such as, by way of non-limiting example, processor(s), storage, sensor(s), I/O system(s), communication system(s), etc.

The processor(s) may comprise one or more sets of electronic circuitries that include any number of logic units, registers, and/or control units to facilitate the execution of computer-readable instructions (e.g., instructions that form a computer program). Such computer-readable instructions may be stored within storage. The storage may comprise physical system memory and may be volatile, non-volatile, or some combination thereof. Furthermore, storage may comprise local storage, remote storage (e.g., accessible via communication system(s) or otherwise), or some combination thereof.

In some implementations, the processor(s) may comprise or be configurable to execute any combination of software and/or hardware components that are operable to facilitate processing using machine learning models or other artificial intelligence-based structures/architectures. Artificial intelligence-based structures/architectures may take on any suitable form, such as by comprising or utilizing hardware components and/or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

In some instances, actions performable by a system may rely at least in part on communication system(s) for receiving information from remote system(s), which may include, for example, separate systems or computing devices, sensors, and/or others. The communications system(s) may comprise any combination of software or hardware components that are operable to facilitate communication between on-system components/devices and/or with off-system components/devices. For example, the communications system(s) may comprise ports, buses, or other physical connection apparatuses for communicating with other devices/components. Additionally, or alternatively, the communications system(s) may comprise systems/components operable to communicate wirelessly with external systems and/or devices through any suitable communication channel(s), such as, by way of non-limiting example, Bluetooth, ultra-wideband, WLAN, infrared communication, and/or others.

A system may comprise or be in communication with sensor(s). Sensor(s) may comprise any device for capturing or measuring data representative of perceivable phenomenon. By way of non-limiting example, the sensor(s) may comprise one or more image sensors, microphones, thermometers, barometers, magnetometers, accelerometers, gyroscopes, and/or others.

Furthermore, a system may comprise or be in communication with I/O system(s). I/O system(s) may include any type of input or output device such as, by way of non-limiting example, a touch screen, a mouse, a keyboard, a controller, and/or others, without limitation. For example, the I/O system(s) may include a display system that may comprise any number of display panels, optics, laser scanning display assemblies, and/or other components. One will appreciate, in view of the present disclosure, that the sensor(s) may, in some instances, be utilized as I/O system(s).

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “computer-readable recording media,” “physical computer storage media,” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” may comprise one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

It is contemplated that various combinations or sub-combinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

We claim:

1. A thrombectomy system, comprising:
one or more processors; and
one or more computer-readable recording media that store instructions that are executable by the one or more processors to configure the thrombectomy system to:
activate a low-level aspiration state or a non-aspiration state, wherein the low-level aspiration state comprises operation of one or more aspiration components;
during operation of the low-level aspiration state or the non-aspiration state, monitor sensor data associated with at least some of the one or more aspiration components;
process the sensor data using a clot engagement analysis module; and
after determining that output of the clot engagement analysis module satisfies one or more conditions, selectively activate a clot removal state, wherein the clot removal state comprises operation of the one or more aspiration components with different parameters than the low-level aspiration state.

2. The thrombectomy system of claim 1, wherein the one or more aspiration components comprise an aspiration catheter, a vacuum pump, a vacuum canister, and suction tubing.

3. The thrombectomy system of claim 1, wherein the low-level aspiration state comprises refraining from activating a high-pressure saline jet at a distal region of an aspiration catheter.

4. The thrombectomy system of claim 1, wherein activation of the clot removal state comprises increasing vacuum pressure at a distal region of an aspiration catheter.

5. The thrombectomy system of claim 1, wherein activation of the clot removal state comprises activating a high-pressure saline jet at a distal region of an aspiration catheter.

6. The thrombectomy system of claim 2, wherein the sensor data comprises pressure data associated with the aspiration catheter, the vacuum canister, and/or the suction tubing.

7. The thrombectomy system of claim 6, wherein the clot engagement analysis module is configured to compare the pressure data to reference pressure data associated with clot engagement or clot removal.

8. The thrombectomy system of claim 6, wherein the pressure data indicates a blood pressure wave, and wherein the clot engagement analysis module is configured to compare the blood pressure wave to a reference blood pressure wave associated with clot engagement.

9. The thrombectomy system of claim 6, wherein the pressure data indicates a change in pressure over time, and wherein the clot engagement analysis module is configured to compare the change in pressure over time to a reference change in pressure over time associated with clot engagement or clot removal.

10. The thrombectomy system of claim 6, wherein the clot engagement analysis module comprises one or more AI modules trained on pressure data of vacuum canisters and/or suction tubing during clot engagement or clot removal.

11. The thrombectomy system of claim 1, the instructions are executable by the one or more processors to further configure the thrombectomy system to:
during operation of the clot removal state, monitor sensor data associated with the at least some of the one or more aspiration components;
process the sensor data using a clot removal analysis module; and
after determining that output of the clot removal analysis module satisfies one or more conditions, selectively deactivate the clot removal state.

12. A thrombectomy system, comprising:
one or more processors; and
one or more computer-readable recording media that store instructions that are executable by the one or more processors to configure the thrombectomy system to:
during operation of a clot removal state that comprises operation of one or more aspiration components, monitor sensor data associated with at least some of the one or more aspiration components;
process the sensor data using a clot removal analysis module; and
after determining that output of the clot removal analysis module satisfies one or more conditions, selectively deactivate the clot removal state.

13. The thrombectomy system of claim 12, wherein the one or more aspiration components comprise a vacuum pump, a vacuum canister, and suction tubing.

14. The thrombectomy system of claim 13, wherein the sensor data comprises pressure data associated with the vacuum canister and/or the suction tubing.

15. The thrombectomy system of claim 14, wherein the clot removal analysis module is configured to compare the pressure data to reference pressure data associated with completion of clot removal.

16. The thrombectomy system of claim 14, wherein the pressure data indicates a change in pressure over time, and wherein the clot removal analysis module is configured to compare the change in pressure over time to a reference change in pressure over time associated with completion of clot removal.

17. A thrombectomy system, comprising:
one or more processors; and
one or more computer-readable recording media that store instructions that are executable by the one or more processors to configure the thrombectomy system to:
during operation of a low-level aspiration state or a clot removal state that comprises operation of one or more aspiration components, monitor flow data obtained by a flow sensor associated with the thrombectomy system;
process the flow data using a flow data analysis module; and
after determining that output of the flow data analysis module satisfies one or more conditions, selectively deactivate the low-level aspiration state or the clot removal state.

18. The thrombectomy system of claim 17, wherein the one or more aspiration components comprise a vacuum pump, a vacuum canister, and suction tubing.

19. The thrombectomy system of claim 18, wherein the flow sensor is connected to the suction tubing.

20. The thrombectomy system of claim 17, wherein the output of the flow data analysis module indicates an amount of fluid aspirated via operation of the one or more aspiration components.
